(19) United States
(12) Patent Application Publication
(10) Pub. No.: US 2021/0290524 A1
(43) Pub. Date: Sep. 23, 2021

(54) TOPICAL TRPV1 ANTAGONISTS AND METHODS AND COMPOSITIONS THEREOF

(71) Applicant: CHILDREN'S MEDICAL CENTER CORPORATION, Boston, MA (US)

(72) Inventors: Clifford J. Woolf, Newton, MA (US); Ajay S. YEKKIRALA, Canton, MA (US); Michael COSTIGAN, Cambridge, MA (US)

(73) Assignee: CHILDREN'S MEDICAL CENTER CORPORATION, Boston, MA (US)

(21) Appl. No.: 16/330,682

(22) PCT Filed: Sep. 5, 2017
(86) PCT No.: PCT/US2017/050032

§ 371 Date: Mar. 5, 2019

Related U.S. Application Data
(60) Provisional application No. 62/383,713, filed on Sep. 6, 2016.

(51) Int. Cl. A61K 9/00; A61K 31/357; A61K 45/06; A61P 23/02

(52) U.S. Cl. CPC A61K 9/0014; A61P 23/02; A61K 45/06; A61K 31/357

(57) ABSTRACT

The technology disclosed herein generally relates to the field of pain relief, and relates to a topical composition comprising a TRPV1 antagonist. The technology disclosed herein provides for methods, compositions and devices for topical application for the treatment of pain, allodynia or hyperalgesia associated with acute tissue injury, e.g., for the treatment of a wounds or tissue injury, including but not limited to incision, abrasion, laceration, puncture and avulsion. The methods, compositions and devices disclosed herein can be used for point-of-care treatment of a tissue injury or wound, e.g., in emergency care for first responders, emergency medical technicians (EMTs), ambulance workers, firemen, in field surgery, combat medicine or casualty care and/or first aid kits.

### CROSS REFERENCED TO RELATED APPLICATIONS

This Application claims the benefit under 35 U.S.C. § 119(e) of U.S. Provisional Application Ser. No: 62/383,713 filed on filed Sep. 6, 2016, the content of which is incorporated herein in its entirety by reference.

### GOVERNMENT SUPPORT

This invention was made with Government Support under NIH Grant PO1NS072040 and R37NS039518 awarded by the National Institutes of Health, and the Government has certain rights in the invention.

### FIELD OF THE INVENTION

The present invention generally relates to the field of pain relief, and relates to a topical composition comprising a TRPV1 antagonist, for example in composition for topical application for the treatment of pain, allodynia or hyperalgesia associated with acute tissue injury, e.g., for the treatment of a wounds or tissue injury, including but not limited to incision, abrasion, laceration, puncture and avulsion.

### BACKGROUND OF THE INVENTION

TRPV1 antagonists for the treatment of various types of pain are being developed. In particular, several companies and academic groups have studied the effectiveness of systemically administered TRPV1 antagonists in relieving various types of pain. However systemic administration of TRPV1 antagonists has wide-spread side effects, for example, frequently leads to hyperthermia. As such, clinical use of TRPV1 antagonists has been halted or significantly hampered as TRPV1 antagonists are normally administered systemically.

Therefore, there is a need to develop compositions for pain management comprising TRPV1 antagonists that have reduced systemic side effects and/or do not lead to hyperthermia.

### SEQUENCE LISTING

The instant application contains a Sequence Listing which has been submitted electronically in ASCII format and is hereby incorporated by reference in its entirety. Said ASCII copy, created on Sep. 1, 2017, is named 701039-087771-PCT_SL.txt and is 17,512 bytes in size.

### SUMMARY OF THE INVENTION

The disclosure herein generally provides methods, compositions and kits for topical application of transient receptor potential vanilloid (TRPV1) antagonists formulated for topical application. In particular, the present invention is based on the surprising discovery that human saliva includes peptides belonging to the opiorphin family that antagonize TRPV1. Moreover, the inventors have demonstrated that Opiorphin was unexpectedly found to be selective for TRPV1 at physiologically relevant concentrations, and that a selective TRPV1 antagonist, e.g., AMG9810 as an exemplary TRPV1 antagonist, surprisingly eliminated the nocifensive licking behavior elicited in response to a wound or tissue injury in mice. Indeed, the inventors surprisingly discovered that wound licking behavior was completely abolished in mice lacking either TRPV1 receptors (e.g., in TRPV1 KO mice) or TRPV1+ neurons (e.g., TRPV1-DTA mice strain which lacking TRPV1+ neurons), further demonstrating that the inventors have discovered the importance of TRPV1 in wound and incision pain. Moreover, in a mouse model in vivo, the inventors demonstrate that an incision injury (e.g., a wound) to the skin resulted in ongoing activity in TrpV1 expressing sensory neurons and that topical TRPV1 agonist capsaicin applied after the acute wound demonstrated an unexpected high number of neurons, ˜84% (32 of 38 neurons) of neurons activated by injury were also TRPV1-positive. Accordingly, the inventors have demonstrated that because the majority of injured neurons are TRPV1-positive. topical application of TRPV1 antagonists work well for pain relief when applied topically to wounds in mammals

Accordingly, the inventors have discovered that a tissue injury, e.g., a wound results in activation of TRPV1 and that endogenous TRPV1 antagonists exist in human and animal saliva, and that topical administration of TRPV1 antagonists can function to provide analgesia or reduce pain sensation of wound or tissue-injury induced pain. Additionally, while the TRPV1 antagonist SB705498 has been applied topically for treatment of itch (Gibson et al., 2013), it was discovered not to be of symptomatic benefit for histaminergic or non-histaminergic induced itch. In contrast, here the inventors have demonstrated a symptomatic effect of a topically applied TRPV1 antagonist that is effective for reducing pain associated with tissue injury or wounds.

Accordingly, aspects of the invention generally relate to a composition comprising: (i) a pharmaceutical composition comprising a transient receptor potential vanilloid (TRPV1) antagonist formulated for topical application; and (ii) an applicator for topical administration of the pharmaceutical composition.

In some embodiments, the TRPV1 antagonist is formulated as a powder, ointment or salve, aerosol, gel, emulsion, foam, cream or lotion. In some embodiments, the applicator is a topical applicator device, for example, a wipe, a spray, pump or aerosol bottle, a dermal patch, a matrix, a brush, a dressing or sponge, a syringe applicator, optionally calibrated, an ampule, a bottle with irrigating nozzle, pipette dropper or liquid dispenser, a topical spreading applicator, a tube, a topical dosing device. In some embodiments, topical administration is epicutaneous (i.e., on the skin), administration directly to a skin or wound, or can be percutaneous (through the skin) or can be via transdermal, or ophthalmic administration, nasal drops, or via vaginal or rectal suppositories. In some embodiments, the pharmaceutical composition comprising the TRPV1 antagonist is present in the applicator, allowing immediate topical administration of the pharmaceutical composition to a subject.

In some embodiments, the pharmaceutical composition comprises at least two or more TRPV1 antagonists. In some embodiments, the pharmaceutical composition further comprises a second therapeutic agent, e.g., a transient receptor potential ankyrin (TRPA1) antagonist to further reduce pain, or any one or more of; an antibiotic, an antimicrobial agent, clotting agent, anti-inflammatory agent or pain management agent and the like suitable for treatment of wounds or injured tissue. In some embodiments, the pharmaceutical composition does not comprise an aromatic alcohol. In some embodiments, the TRPV1 antagonist is stable at room temperature.

In some embodiments, a TRPV1 antagonist is formulated for increased permeability into the skin, and in some embodiments, a TRPV1 antagonist is formulated for controlled or sustained release. In some embodiments, a TRPV1 antagonist is topically administered at a lower unit dose than the unit dose used for systemic administration, by oral or enteral administration or parenteral administration of the same TRPV1 antagonist. In some embodiments, a topical delivery device or applicator is capable of administering a unit dose of the TRPV1 antagonist which is sufficient to reduce the acute or nociceptive pain sensation in the subject with a tissue injury.

In some embodiments, the methods, compositions and kits are useful for the immediate treatment of acute pain or nociceptive pain of a subject in the field, or at a location where the subject was injured, e.g., for treatment of pain within 0 hours and 3 hours of surgery, an accident, or the occurrence of an injury, tissue injury, trauma or wound to the subject.

Another aspect of the present invention relates to a kit comprising (i) a pharmaceutical composition comprising a transient receptor potential vanilloid (TRPV1) antagonist formulated for topical application; and (ii) an applicator for topical administration of the pharmaceutical composition, and optionally, the kit comprises instructions for use by a non-medically trained person.

Another aspect of the present invention relates to a method for treating acute pain or nociceptive pain in a subject, comprising topically administering at, or near a site of tissue injury in the subject, a pharmaceutical composition comprising a TRPV1 antagonist. In some embodiments, the method TRPV1 antagonist is formulated for topical application, and can be topically administered to the subject between 0 hours and 3 hours of the occurrence of tissue injury. In some embodiments, the topical administration is epicutaneous, administration directly to a tissue injury, e.g., wound. In some embodiments, the topical administration is transdermal, ophthalmic, nasal drops, vaginal, rectal suppositories. In some embodiments, the tissue injury is a burn. In alternative embodiments, the tissue injury is not a burn. In some embodiments, the methods relate to the treatment of a tissue injury which is a surgical incision, a wound, or tissue trauma.

In some embodiments, the methods relate to the treatment of acute pain or nociceptive pain, which can be post-surgical incision pain.

In some embodiments, the pharmaceutical composition comprising the TRPV1 antagonist is not washed out or rinsed from the site of the tissue injury. In some embodiments, the pharmaceutical composition comprising a TRPV1 antagonist is topically administered to the skin immediately prior to a surgical incision.

In some embodiments, the methods, compositions and kits are useful in the treatment of a human subject, a domestic animal or a commercial animal.

The compositions, methods and devices described herein for topical application of a TRPV1 antagonist to a tissue injury or wound has multiple advantages as opposed to classical oral or systemic administration of a TRPV1 antagonist in that local application of the TRPV1 antagonist, such as: it is applied only on the pain area where relief is needed, there is absence of systemic side effects, the TRPV1 antagonist can be used as lower concentrations without side effects and the same therapeutic effect, or alternatively, at higher concentrations of TRPV1 antagonist at the area of pain or allodynia, fast onset of action, low or no systemic drug levels, absence of drug-drug interactions and improvement of compliance, as well as very low risk of dependency or abuse.

### BRIEF DESCRIPTION OF FIGURES

FIGS. 1A-1C show human saliva inhibits capsaicin-induced calcium flux in sensory neurons. FIG. 1A shows whole saliva collected from various different human donors significantly inhibited capsaicin-induced calcium flux in primary murine dorsal root ganglia (DRG) neurons. FIG. 1B shows a chromatograph of human saliva samples. FIG. 1C shows that 7 of 37 fractions (highlighted with an arrow) produced significant inhibition (p<0.05, One-way ANOVA with Bonferonni post-hoc test) of capsaicin-induced calcium flux in HEK-293 cells transiently expressing rTRPV1.

FIGS. 2A-2G shows the human salivary pentapeptide opiorphin inhibits TRPV1. FIG. 2A shows the structure and space-filling model of opiorphin pentapeptide QRFSR (SEQ ID NO: 3). FIG. 2B shows Opiorphin inhibited capsaicin-induced calcium flux in primary mouse DRG neurons (384-well plate-based imaging) (Mean±SEM, n=12-16). FIG. 2C shows whole cell patch-clamping of DRG neurons revealed that co-application of opiorphin (1 μM) with capsaicin (1 μM) potently and reversibly eliminated capsaicin-evoked currents (n=6). FIG. 2D shows that Opiorphin (1 μM) inhibited capsaicin-evoked currents even 10 s into capsaicin (1 μM) application. FIG. 2E shows Pertussis toxin (PTX, 1 μg/mL, 3 h) did not reduce opiorphin's actions on capsaicin-induced currents. FIG. 2F shows Opiorphin inhibited capsaicin-induced currents in HEK293 cells transiently expressing hTRPV1. FIG. 2G shows Opiorphin (1 μM) inhibited capsaicin-evoked (1 μM) currents at all voltages tested in HEK-293 cells transiently expressing rTRPV1.

FIGS. 3A-3G show that opiorphin inhibits TRPV1 via the external pore turret region between the S5 and S6 helices. FIG. 3A shows that Opiorphin is a non-competitive antagonist concentration response profile with capsaicin in HEK-293 cells transiently expressing rTRPV1 (n=3). FIGS. 3B and 3C shows that external but not internal application of opiorphin leads to inhibition of TRPV1 (n=6 cells). FIG. 3D shows that Opiorphin's antagonistic activity was eliminated in deletion and substitution mutants of the external pore turret region of TRPV1 expressed in HEK-293 cells (n=3). FIG. 3E shows that Opiorphin (1 μM) significantly inhibited currents evoked by protons (pH 5.6). FIGS. 3F and 3G shows that surface representation of TRPV1 (PDB code 3j5p) viewed from the extracellular face (top view) (FIG. 3F) or the side (FIG. 3G). In the side view, the approximate position of the membrane bilayer is indicated with black lines. Residues 603-627, corresponding to the turrets, were modeled as yellow loops of varying structure in each subunit, to provide a sense of scale. Two opiorphin peptides, modeled here as extended strands, were placed over the channel pore (green spheres) or in the groove between two S1-S4 bundles (blue spheres), respectively. Each peptide illustrates a possible binding mode that could stabilize a closed state of the channel The stoichiometry of binding (e.g. one per subunit or one per channel) remains to be determined.

FIGS. 4A-4F shows that opiorphin inhibits pain-related and wound-licking behavior in mice via TRPV1. FIG. 4A shows that opiorphin significantly reduced the acute pain-like responses induced by capsaicin (p<0.05, n=7 per group) and FIG. 4B shows reduced the thermal hypersensitivity (Hot-plate test (52±0.1° C.), p<0.001, n=7-8 per group) and FIG. 4C shows mechanical hypersensitivity (Von frey filaments, p<0.0001, n=9 per group) that follows intraplantar capsaicin injection. FIG. 4D shows topical application of opiorphin (10 μM, 5 s) eliminated the licking/biting response to superficial paw incision in C57 mice (p=0.0006, n=14) as did the TRPV1 selective antagonist, AMG 9810 (30 μM) (p=0.0379, n=9) when compared with topical saline application (n=17). FIG. 4E shows mice lacking TRPV1+ neurons show attenuated wound licking response, and FIG. 4F show mice lacking TRPV1 receptors show attenuated wound licking response (n=9-11).

FIGS. 5A-5C show TRPV1 inhibition by opiorphin family peptides. FIG. 5A shows that Sialoprhin, but not SMR3 peptide (FIG. 5B), inhibits capsaicin-induced calcium currents in DRG neurons. FIG. 5C shows control recording of a DRG neuron where consecutive application of capsaicin shows the expected level of desensitization, but significantly less than the inhibition produced by sialorphin in FIG. 5A or opiorphin, as shown in FIG. 2E.

FIG. 6 shows that opiorphin inhibits TRPV1. Current-voltage relationship in HEK293 cells transiently expressing rTRPV1 showing that opiorphin (1 uM) significantly inhibits Resiniferatoxin (RTX, 100 nM) at all voltages tested.

FIGS. 7A-7D show that opiorphin is a selective antagonist for TRPV1. FIG. 7A shows that opiorphin (30 μM) does not block calcium flux induced by AITC (TRPA1 agonist, 50 μM, 30 s), FIG. 7B shows that Menthol (TRPM8 agonist, 100 μM, 15 s), FIG. 7C shows GSK 1016790A (TRPV4 agonist, 1 μM, 15 s) or FIG. 7D shows α,β-methylene-ATP (P2×3 agonist, 30 μM, 30 s) in DRG neurons.

FIGS. 8A-8B shows IHC of salivary glands. FIG. 8A shows immunostaining of PROL1, the protein containing opiorphin, and shows that it is expressed in the submandibular gland (SMG) and not in the sublingual or parotid glands. FIG. 8B shows immunostaining of PROL1 and shows that the PROL1 antibody strongly stains the acinar cells of the SMG (left panel) and this staining is absent when preincubated with the opiorphin native peptide (right panel).

FIG. 9A-9C shows that incision injury generates ongoing activity in TrpV1 expressing afferents. FIG. 9A is a micrograph of calcium imaging field in trigeminal ganglion of VGlut2^{Cre}; Rosa26^{LSL-GCαMP6f} mouse, with neurons active after injury highlighted. Scale bar is 100 μm. FIG. 9B shows GCaMP fluorescence traces of neurons shown in FIG. 9A, showing injury-evoked activity and responses to topical stimulation with vehicle (VEH, 2% ethanol) and 5 mM capsaicin (CAPS). FIG. 9C shows the distribution of TRPV1 mediated responses in injury-activated neurons; 32 of 38 neurons (84%, CAPS+) recorded from 5 VGlut2^{Cre}; Rosa26^{LSL-GCαMP6f} animals responded to stimulation with capsaicin but not vehicle. (The VGlut2^{Cre}; Rosa26^{LSL-GCαMP6f} mouse is generated by crossing a VGlut2-Cre mice with a td-Tomato mice, therefore the resulting double transgenic VGlut2^{Cre}; Rosa26^{LSL-GCαMP6f} mice express tD-Tomato (a red fluorescence protein) in all the sensory neurons but not in any other type of neuron. This allows for labeling and imaging of only sensory neurons in vivo.)

### DETAILED DESCRIPTION

As disclosed herein, one aspect of the present invention relates to methods, compositions and kits for topical application of transient receptor potential vanilloid (TRPV1) antagonists formulated for topical application to mammals, e.g., humans and for veterinary applications. In particular, the present invention allows topical application of TRPV1 antagonists, e.g., in the field, and/or immediately after a tissue injury or wound.

Provided herein do compositions, methods and kits for topical administration of a TRPV1 antagonist for treating or preventing pain, e.g., after a wound or incision, in a subject comprise topically administering to the subject a transient receptor potential (TRP) ion channel inhibitor. In some embodiments, the pain is a result of an incision or wound. In some embodiments, the TRP ion channel inhibitor inhibits transient receptor potential vanilloid (TRPV1). In alternative embodiments, the TRP ion channel inhibitor inhibits TRP ankyrin (TRPA1). Thus, provided is a method for reducing pain in a subject comprising topically administering to the subject an agent that inhibits the activity or expression a transient receptor potential (TRP) ion channel inhibitor. Also provided is a method for reducing or preventing pain in a subject comprising administering to the subject an agent that inhibits the activity or expression a TRP ion channel inhibitor. Optionally, the method comprises selecting a subject in need of relief of pain. Optionally, the subject is under anesthesia. Optionally, the method further comprises selecting a subject under anesthesia. Optionally, the TRP is transient receptor potential vanilloid (TRPV1) and TRP ankyrin (TRPA1). Optionally, the inhibitor binds the TM5 domain of TRPA1. Optionally, the inhibitor binds SEQ ID NO:1 or SEQ ID NO:2. Optionally, the pain is associated with administration of an anesthetic to the subject. Optionally, the subject is a surgical patient. Optionally, the pain is post-surgical pain. Optionally, the TRP inhibitor is administered at the same time, before or after an anesthetic is administered to the subject.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, a transient receptor potential vanilloid (TRPV) ion channel refers to transient receptor potential vanilloid (TRPV) and homologs, variants and isoforms thereof. There are a variety of sequences that are disclosed on Genbank, at www.pubmed.gov, and these sequences and others are herein incorporated by reference in their entireties as well as for individual subsequences contained therein. For example, the amino acid and nucleic acid sequences of human TRPV 1 can be found at GenBank Accession Nos. NP 542436.2 (SEQ ID NO: 1) and NM 080705.3, respectively. For TRP ankyrin (TRPA1): the amino acid and nucleic acid sequences of human TRPA1 can be found at GenBank Accession Nos. NP 015628.2 (SEQ ID NO: 2) and NM007332.2, respectively.

The term “TRPV1 antagonist” and “TRPV1 inhibitor” includes any agent that can inhibit the activity of TRPV1 (i.e. block TRPV1-mediated signaling cascade) at a physiologically relevant concentration.

As used herein, “pain” includes nociception and the sensation of pain, and pain can be assessed objectively and subjectively, using pain scores and other methods, e.g., with protocols well-known in the art. Post-surgical pain, as used herein, includes allodynia (i.e., pain due to a stimulus that does not normally provoke pain) and hyperalgesia (i.e., increased response to a stimulus that is normally painful), which can in turn, be thermal or mechanical (tactile) in nature. In some embodiments, the pain is characterized by thermal sensitivity, mechanical sensitivity and/or resting pain (e.g. persistent pain in the absence of external stimuli). In some embodiments, the post-surgical pain includes mechanically-induced pain or resting pain. In other embodiments, the post-surgical pain includes resting pain. The pain can be primary (e.g., resulting directly from the pain-causing event) or secondary pain (e.g., pain associated with, but not directly resulting, from the pain-causing event).

The term “tissue injury” refers to an internal or external break in body tissue, or damage to the skin and/or tissues that connect, support, or surround other structures and organs of the body. The term “tissue injury” also refers to a break in body tissue, typically involving the skin.

The term “wound” refers to an injury to living tissue caused by a cut, blow, or other impact. A wound typically refers to an injury where the skin is cut or broken, or involves laceration or breaking of a membrane (such as the skin) and usually damage to underlying tissues. Types of wounds include, but are not limited to: abrasions, incisions, laceration, punctures and avulsions.

The term “topical administration” as used herein refers to applying to, or affecting, a localized area of the body. The term “topical administration” also refers to application of the composition comprising a TRPV1 antagonist to a specific area on, or in the body, rather than taken internally or injected. In some embodiments, topical administration of a composition comprising a TRPV1 antagonist is applied on the skin surrounding a wound, or in the wound, or at, or near a target site, e.g., at or in close proximity to a tissue injury, and can be, in some embodiments, on the exposed surface of the wound (e.g., where the skin is broken and/or the underlying tissues).

The term “topical analgesic” as used herein refers to a composition applied topically that is capable of producing analgesia, i.e., one that relieves pain by altering perception of nociceptive stimuli without producing anesthesia or loss of consciousness, or reduces the subjects response to painful stimuli.

The term “functional derivative” and “mimetic” are used interchangeably, and refers to compounds which possess a biological activity (either functional or structural) that is substantially similar to a biological activity of the entity or molecule for which it's a functional derivative. The term functional derivative is intended to include the fragments, variants, analogues or chemical derivatives of a molecule.

As used throughout, the term “subject” is meant an individual. Thus, the subject can include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject can be a mammal such as a primate or a human. The term subject also includes individuals of different ages. Thus, a subject includes an infant, child, teenager or adult. The term “animal,” includes, but is not limited to, a cow, monkey, baboon, chimpanzee, horse, sheep, pig, chicken, turkey, quail, cat, dog, mouse, rat, rabbit, and guinea pig.

The phrase “pharmaceutically acceptable salt”, as used herein, is any pharmaceutically acceptable salt that can be prepared from a TRPV1 antagonist including a salt formed from an acid and a basic functional group, such as a nitrogen group, of a TRPV1 antagonist compound. Illustrative salts include, but are not limited, to sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, -toluenesulfonate, and pamoate (i.e., 1,1-methylene-bis-(2-hydroxy-3-naphthoate)) salts.

The term “pharmaceutically acceptable salt” also includes a salt prepared from a TRPV1 antagonist as disclosed herein having an acidic functional group, such as a carboxylic acid functional group, and a pharmaceutically acceptable inorganic or organic base. Suitable bases include, but are not limited to, hydroxides of alkali metals such as sodium, potassium, cesium, and lithium; hydroxides of alkaline earth metal such as calcium and magnesium; hydroxides of other metals, such as aluminum and zinc; ammonia and organic amines, such as unsubstituted or hydroxy-substituted mono-, di-, or trialkyl amines; dicyclohexylamine; tributyl amine; pyridine; picoline; N-methyl-iv-ethyl amine; diethylamine; triethylamine; mono-, bis-, or tris-(2-hydroxy-(Ci-C3)alkyl amines), such as mono-, bis-, or tris-(2-hydroxyethyl)amine, 2-hydroxy-feri-butylamine, or tris-(hydroxymethyl)methylamine, NN-di-[(Ci-C3)alkyl]-N-(hydroxy-(Ci-C3)alkyl)-amines, such as N,N-dimethyl-N-(2-hydroxyethyl)amine, or tri-(2-hydroxyethyl)amine; iV-methyl-D-glucamine; and amino acids such as arginine, lysine, and the like. In one embodiment, the pharmaceutically acceptable salt is a hydrochloride-salt, a sulfate-salt, a sodium-salt, a potassium-salt, a benzene sulfonic acid-salt, an ara-toluenesulfonic acid-salt, or a fumaric acid-salt. In another embodiment, the pharmaceutically acceptable salt is a hydrochloride-salt or a sulfate-salt. In another embodiment, the pharmaceutically acceptable salt is a hydrochloride-salt. In another embodiment, the pharmaceutically acceptable salt is a sulfate-salt. In another embodiment, the pharmaceutically acceptable salt is a sodium-salt. In another embodiment, the pharmaceutically acceptable salt is a potassium-salt. In another embodiment, the pharmaceutically acceptable salt is a para-toluenesulfonic acid-salt. One skilled in the art will recognize that, e.g., acid addition salts, of a TRPV1 antagonist can be prepared by reaction of the compounds with the appropriate acid by a variety of known methods.

The term “Solvates” are known in the art and are considered to be a combination, physical association and/or solvation of a TRPV1 antagonist with a solvent molecule. This physical association can involve varying degrees of ionic and covalent bonding, including hydrogen bonding. When the solvate is of the stoichiometric type, there is a fixed ratio of the solvent TRPV1 antagonist e.g., a disolvate, monosolvate or hemisolvate when the solvent molecule: TRPV1 antagonist molecule molar ratio is 2:1, 1:1 or 1:2, respectively. In other embodiments, the solvate is of the nonstoichiometric type. For example, a TRPV1 antagonist crystal can contain solvent molecules in the structural voids, e.g., channels, of the crystal lattice. In certain instances, the solvate can be isolated, for example when one or more solvent molecules are incorporated into the crystal lattice of a crystalline solid. Thus, “solvate”, as used herein, encompasses both solution-phase and isolatable solvates. A TRPV1 antagonist can be present as a solvated form with a pharmaceutically acceptable solvent, such as water, methanol, ethanol, and the like, and it is intended that the disclosure include both solvated and unsolvated TRPV1 antagonist forms. As “hydrate” relates to a particular subgroup of solvates, i.e., where the solvent molecule is water, hydrates are included within the solvates of the disclosure. In one embodiment, a TRPV1 antagonist is present as a monohydrate, i.e., as a free base where the water: TRPV1 antagonist molar ratio is about 1:1 , e.g., from 0.91:1 to 1.09:1 in one embodiment, from 0.94:1 to 1.06:1 in another embodiment, from 0.97:1 to 1.03:1 in another embodiment, and from 0.985:1 to 1.015:1 in another embodiment, each said embodiment taking no account of surface water that might be present, if any. Preparation of solvates is known in the art. For example, Caira et al, “Preparation and Crystal Characterization of a Polymorph, a Monohydrate, and an Ethyl Acetate Solvate of the Antifungal Fluconazole,” J. Pharmaceut. Sci., 93(3):601 -611 (2004), describes the preparation of solvates of fluconazole with ethyl acetate and with water. Similar preparations of solvates, hemisolvate, hydrates, and the like are described by Van Tonder et al., “Preparation and Physicochemical Characterization of 5 Niclosamide Solvates and 1 Hemisolvate,” AAPS Pharm. Sci. Tech., 5(1):Article 12 (2004), and Bingham et al, “Over one hundred solvates of sulfathiazole,” Chem. Comm., pp. 603-604 (2001).

The phrase “effective amount,” when used in connection with a TRPV1 antagonist means an amount effective for: (a) treating a pain condition or symptom thereof; and/or (b) detectably inhibiting TRPV1 receptor function in a cell.

The phrase “effective amount,” when used in connection with another therapeutic agent or a second therapeutic agent means an amount for providing the therapeutic effect of the second therapeutic agent when the second therapeutic agent is used alone, or in combination with the TRPV1 antagonist. In some embodiments, the effective amount of the second therapeutic agent when used with a TRPV1 antagonist is less than the effective amount of the second therapeutic agent when used alone, or not in the presence or combination with a TRPV1 antagonist. In some embodiments, a TRPV1 antagonist and second therapeutic agent are synergistic, in that they work together such that one agent increases the effectiveness of the other. For example, the topical application of a TRPV1 antagonist increases the effectiveness of the topical application of the secondary therapeutic agent, such that the effective dose of a topically applied secondary therapeutic agent is lower in the presence of the TRPV1 antagonist, and higher in the absence of the TRPV1 antagonist. In some embodiments, the combined analgesic effect of a topically applied TRPV1 antagonist and topically applied second therapeutic agent is greater than when each of these agents are used alone. Synergistic effects are typically detected when the TRPV1 antagonist and second therapeutic agent work by different mechanisms so together they form a stronger analgesic effect than then they are used individually. In some embodiments, a TRPV1 antagonist and second therapeutic agent are additive, in that combined analgesic effect of a topically applied TRPV1 antagonist and topically applied second therapeutic agent is equal to the sum of the analgesic effect when these two agents are used alone. Additive effects are typically detected when the TRPV1 antagonist and second therapeutic agent work by the same or similar mechanism.

The phrase “therapeutic index,” describes the gap between the dose that is effective, and the dose that induces adverse effects.

The terms “modulate”, “modulating”, and the like as used herein with respect to the TRPV1 receptor mean the mediation of a pharmacodynamic response (e.g. analgesia) in an animal from (i) inhibiting or activating the receptor, or (ii) directly or indirectly affecting the normal regulation of the receptor activity. Compounds that modulate the receptor activity include antagonists, mixed agonists/antagonists, mixed partial agonists/antagonists and compounds which directly or indirectly affect regulation of the receptor activity.

As used herein the terms “treatment”, “treat” or “treating” and the like refers to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, a method for treating pain or a disease associated with pain is considered to be a treatment if there is a 10% reduction in one or more symptoms of the disease, e.g., a reduction of the sensation of pain in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition. Therefore, the terms include the amelioration or cessation of the sensation of pain or a symptom thereof. In one embodiment, treating includes inhibiting, for example, decreasing the overall sensation or frequency of episodes of pain or a symptom thereof.

The term “Reducing pain” includes a decrease in pain and does not require complete alleviation of pain r symptoms, and does not require a cure. In various embodiments, reducing pain includes even a marginal decrease in pain. By way of example, the topical administration of an effective dosage of a TRPV1 antagonist may be used to prevent, treat or relieve the symptoms of pain for different diseases or conditions.

As used herein, the terms prevent, preventing and prevention of a disease or disorder refers to an action, for example, administration of a therapeutic agent, that occurs before a subject begins to suffer from one or more symptoms of the disease or disorder, which inhibits or delays onset of the severity of one or more symptoms of the disease or disorder. As used herein, references to decreasing, reducing, or inhibiting include a change of 10, 20, 30, 40, 50, 60, 70, 80, 90 percent or greater as compared to a control level. Such terms can include but do not necessarily include complete elimination.

The articles “a” and “an” are used herein to refer to one or to more than one (i.e., to at least one) of the grammatical object of the article. By way of example, “an element” means one element or more than one element.

While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

### TRPV1 Antagonists

The transient receptor potential cation channel subfamily V member 1 (TRPV1), also known as the capsaicin receptor and the vanilloid receptor 1, is a protein that, in humans, is encoded by the TRPV1 gene. The vanilloid receptor TRPV1 is a homotetrameric, non-selective cation channel abundantly expressed in the nociceptors (c-fibers). See Caterina M J, Schumacher M A, Tominaga M, Rosen T A, Levine J D, Julius D (October 1997). “The capsaicin receptor: a heat-activated ion channel in the pain pathway”. Nature 389 (6653): 816-24. and Xue Q, Yu Y, Trilk S L, Jong B E, Schumacher M A (August 2001). “The genomic organization of the gene encoding the vanilloid receptor: evidence for multiple splice variants”. Genomics 76 (1-3): 14-20. The function of TRPV1 is detection and regulation of body temperature. Additionally, TRPV1 provides sensation of heat and pain. When TRPV1 is activated by agonists such as capsaicin and other factors such as heat and acidosis, calcium enters the cell and pain signals are initiated. After disease or injury, cutaneous nociceptors may become persistently hyperactive, spontaneously transmitting excessive pain signals to the spinal cord in the absence of painful stimuli, resulting in various types of chronic pain.

TRPV1 antagonists for use in the compositions, kits, and methods as disclosed herein are well known in the art. In some embodiments, TRPV1 antagonists useful in the methods and compositions as disclosed herein include, but are not limited to, fused azabicyclic, heterocyclic, and amide compounds as described, for example, in U.S. Patent Application No. 2004/0157849, U.S. Patent Application No. 2004/0209884, U.S. Patent Application No. 2005/0113576, International Patent Application No. WO 05/016890, U.S. Patent Application No. 2004/0254188, U.S. Patent Application No. 2005/0043351, International Patent Application No. WO 05/040121, U.S. Patent Application No. 2005/0085512, and Gomtsyan et al., 2005, J. Med. Chem. 48:744-752; fused pyridine derivatives as described, for example, in U.S. Patent Application No. 2004/0138454; pyridyl piperazinyl ureas as described, for example, in Swanson et al., 2005, J. Med. Chem. 48:1857-1872 and U.S. Patent Application No. 2005/0049241, as well as AMG8163 (Bannon et al., 2005, 11.sup.th World Congress on Pain) and BCTC (Sun et al., 2003, Chem. Lett. 13:3611-3616); 2-(piperazine-1-yl)-1H-Benzimidazole; pyridazinylpiperazines; urea derivatives as describe, for example, in U.S. Patent Application No. 2005/0107388, U.S. Patent Application No. 2005/0187291, and U.S. Patent Application No. 2005/0154230, as well as A-425619 (El Kouhen et al., 2005, J. Pharmacol. Exp. Ther. 314:400-409); cinnamides, including SB-366791 (Gunthorpe et al., 2004, Neuropharmacology 46:133-149) and AMG 9810 (Gawa et al., 2005, J. Pharmacol. Exp. Ther. 313:474-484); each of which is incorporated by reference.

In some embodiments, TRPV1 antagonists useful in the methods and compositions of as disclosed herein include, for example, TRPV-1 antagonists include capsazepine, (E)-3-(4-t-butylphenyl)-N-(2,3-dihydrobenzo[b][1,4]dioxin-6-yl)acrylamide (commercially available for example as AMG9810 from Tocris Bioscience, Bristol, United Kingdom), and 4-tertiary butyl cyclohexane (commercially available as SYMSITIVE 1609 from Symrise GmbH of Holzminden, Germany, as well as TRPV1 antagonists as disclosed in U.S. Pat. Nos. 8,815,930, 6,933,311, 7,767,705 and U.S. Pat. App. Pub. Nos. 2010/0249203 and 2011/0104301, International Application WO/2008/013861, which are incorporated herein in their entirety by reference.

In some embodiments, TRPV1 antagonists useful in the methods and compositions and devices as disclosed herein include AMG-517 and AMG-628 (Amgen Inc., Thousand Oaks, Calif.). TRPV1 antagonists useful in the present application are also described, for example, in International Patent Application No. WO 2006065484; International Patent Application No. WO 2003070247; U.S. Patent Application No. US 2005080095; and International Patent Application No. WO 2005007642. These disclosures are herein incorporated by reference. Additional TRPV1 antagonists useful in the methods and compositions and devices as disclosed herein include, but are not limited to TRPV1 antagonists: ABT-102, AMG8562, AMG9810, BCTC, SB366791, JNJ17203212, I-TTX, JYL-1421, A-425619, and TRPV1 antagonists N-[4-[6-[4(Trifluoromethyl)phenyl)pyrimidin-4-yloxy]benzothiazol-2-yl]acetamide (also known as AL-49975 or AMG-517), or (R)—N-(4-(6-(4-(1-(4-fluorophenyl)ethyl)piperazin-1-yl)pyrimidin-4-yloxy)benzo[d]thiazol-2-yl)acetamide (AL-49976, also known as AMG-628).

Other TRPV1 antagonists useful in the methods and compositions and devices as disclosed herein are those that have a low inhibitory activity against CYP3A4, such as, e.g., 1-(2-(3,3-dimethylbutyl)-4-(trifluoromethyl)benzyl)-3-(1-methyl-1H-in-dazol-4-yl)urea; methyl 2,2-dimethyl-4-(2-((3-(1-methyl-1H-indazol-4-yl)ureido)methyl)-5-(trifluo-romethyl)phenyl)butanoate; 1-(2-(4-hydroxy-3,3-dimethylbutyl)-4-(trifluoromethyl)benzyl)-3-(1-methyl- -1H-indazol-4-yl)urea; 2,2-dimethyl-4-(2-((3-(1-methyl-1H-indazol-4-yl)ureido)methyl)-5-trifluor-omethyl)phenyl)butanoic acid; 1-[4-Chloro-3-(3,3-dimethylbutyl)benzyl]-3-(1-methyl-1H-indazol-4-yl)urea-; 1-(2-isobutyl-4-(trifluoromethyl)benzyl)-3-(1-methyl-1H-indazol-4-yl)urea; 1-(2-isopropyl-4-(trifluoromethyl)benzyl)-3-(1-methyl-1H-indazol-4-yl)urea; and 1-(4-Chloro-3-isopropylbenzyl)-3-(1-methyl-1H-indazol-4-yl)urea.

TRPV1 antagonists for use in the compositions, kits, and methods as disclosed herein also include those described in Chen et al., Journal of Biomolecular Screening, Vol. 12, No. 1, 61-69 (2007), Bruce R. Bianchi, Robert B. Moreland, Connie R. Faltynek, Jun Chen. ASSAY and Drug Development Technologies. Jun. 1, 2007, 5(3): 417-424, which are incorporated herein in their entirety by reference. Other TRP inhibitors include, but are not limited to, wortmannin, camphor, phosphatidylinositol-4,5-bisphosphate (PIP2), high levels of menthol, AP18, cannabinoids such as WIN 55, 212-2, HC-030031, gadolinium, ruthenium red, capsazepine, AMG 517, SB366791, lodo-resiniferatoxin, resiniferatoxin, LJO-328, and SC0030. See Karashima et al., “Modulation of the transient receptor potential channel TRPA1 by phosphatidylinositol 4,5-biphosphate manipulators,” Pflügers Archiv European Journal of Physiology (2008); Kim et al., “Inhibition of Transient Receptor Potential A1 by Phosphatidylinositol-4,5-bisphosphate,” Am J Physiol Cell Physiol. (2008); Karashima, et al., The Journal of Neuroscience, 2007, 27(37):9874-9884; Patwardhan et al., PNAS, 2006, vol. 103, no. 30, 11393-11398; Taylor-Clarke et al., Mol. Pharmacol. 2008, 73(2):274-81; WO/2008/013861; Bang et al., European Journal of Neuroscience, Volume 26 Issue 9 Page 2516-2523, 2007; Gunthorpe and Szallasi, Curr Pharm Des. 2008; 14(1):32-41; Anderson et al., Thorax. 2004; 59: 730-731; Johansen et al., Toxicological Sciences 89(1):278-86 (2006); and Veronesi et al., Toxicol Sci. 2006 January; 89(1):1-3, which are incorporated herein by reference in their entireties.

In some embodiments, a TRPV1 antagonist for use in the methods, compositions and kits as disclosed herein inhibits the calcium flux into a cell expressing TRPV1 by at least about 12%, or at least about 20%, preferably at least about 50%, such as from about 50% to about 80%, as measured by Calcium Flux Test.

The Calcium Flux Test is performed in the following manner: Recombinant HEK293 cells expressing human vanilloid receptor-1 (TRPV1; accession number AF196175, EMD Millipore, Billerica, Mass.) are grown in D-MEM/F-12 media (Life Technologies, Grand Island, N.Y.) supplemented with 10% fetal bovine serum, 1% non essential amino acids and 400 μg/mL Geneticin (Gibco, Life Technologies). Cells are plated at a concentration of 15,000 cells per well on poly-D-lysine-coated 384 well plates and incubated overnight at 5% CO₂ and 37° C. After incubation, culture media is removed from the wells and replaced with 50 μL per well of Fluo-8 No Wash dye solution (#36315, AAT Bioquest, Sunnyvale, Calif.). The Fluo-8 dye solution is prepared by mixing 20 uL of Fluo-8 NW with 30 mL of 0.33.times. Pluronic™. F127 Plus (BASF of Ludwigshafen, Germany) in assay buffer (1×HBSS+2% of HEPES). Fluo-8 dye is incubated for 30 minutes at room temperature. Intracellular calcium (Ca²⁺) flux is monitored upon exposure to aromatic alcohol in DMSO vehicle by measuring fluorescence intensity at Ex/Em 490/525 on a Functional Drug Screening System (FDSS; Hamamatsu, Germany). Measurements are taken every second for a period of 4 minutes. Results are presented as mean±standard deviation of maximum relative fluorescence units (RFU) during the 4 minute period. Statistical differences are obtained using One Way ANOVA with Tukey Post-hoc test; statistical significance is defined as P.ltoreq.0.05.

In alternative embodiments, the functional activity of a TRPV1 antagonist can be determined using a Ca²⁺ influx assay and measurement of intracellular Ca²⁺ levels ([Ca²⁺]ᵢ) as described below. For example, TPRV1 antagonist compounds can be tested over an 11-point half-log concentration range. Compound solutions are prepared in D-PBS (4× final concentration), and diluted serially across 96-well v-bottom tissue culture plates using a Biomek 2000 roboptic automation workstation (Beckman-Coulter, Inc., Fullerton, Calif.). A 0.2 μM solution of the TRPV1 agonist capsaicin was also prepared in D-PBS. The fluorescent Ca²⁺ chelating dye fluo-4 was used as an indicator of the relative levels of [Ca²⁺]ᵢ in a 96-well format using a Fluorescence Imaging Plate Reader (FLIPR) (Molecular Devices, Sunnyvale, Calif.). Cells were grown to confluency in 96-well black-walled tissue culture plates. Then, prior to the assay, the cells were loaded with 100 μL per well of fluo-4 AM (2 μM, in D-PBS) for 1-2 hours at 23° C. Washing of the cells was performed to remove extracellular fluo-4 AM (2×1 mL D-PBS per well), and afterward, the cells were placed in the reading chamber of the FLIPR instrument. 50 μL of the compound solutions were added to the cells at the 10 second time mark of the experimental run. Then, after a 3 minute time delay, 50 μL of the capsaicin solution was added at the 190 second time mark (0.05 μM final concentration) (final volume=200 μL) to challenge the TRPV1 receptor. Time length of the experimental run was 240 seconds. Fluorescence readings were made at 1 to 5 second intervals over the course of the experimental run. The peak increase in relative fluorescence units (minus baseline) was calculated from the 190 second time mark to the end of the experimental run, and expressed as a percentage of the 0.05 μM capsaicin (control) response. Curve-fits of the data were solved using a four-parameter logistic Hill equation in GraphPad Prism™. (GraphPad Software, Inc., San Diego, Calif.), and IC₅₀ values were calculated.

In some embodiments, a TRPV1 antagonist as disclosed herein is an antagonists of the transient receptor potential vanilloid-1 (TRPV1) receptor with IC₅₀ from 1000 nM to 0.1 nM. In a preferred range, compounds tested had IC₅₀ from 500 nM to 0.1 nM. In a more preferred range, compounds tested had IC₅₀ from 50 nM to 0.1 nM.

TRPV1 antagonists useful in the methods of the invention are also described, for example, in WO 2006065484; WO 2003070247; US 2005080095; WO 2005007642; WO 2003080578; WO2004007459; WO 2006063178; WO 2006062981; WO 2006065646; WO 2006122250; WO2007050732; WO 2005077938; WO 2005077944; WO 2004014871; US 2003195201; US2004152690; WO 2003099284; WO 2004072068; WO 2006044527; WO 2002016318; WO2002016317; WO 2006098554; WO 2006101318; WO 2006101321; WO 2007063925; WO 2006033620; WO 2006038871; WO 2006068592; WO 2006068593; WO 2006068618; WO 2004089881; WO 2007073303; WO 2007091946; WO 2007091948; WO 2007091947; WO 2003014064; WO 2003055848; WO 2003055484; WO 2004072020; WO 2005040119; WO 2005044786; WO 2005044802; WO 2005103018; WO 2006080821; WO 2005003084; WO 2004035533; WO 2003066595; WO 2003074520; WO 2004002983; WO 2004011441; WO 2004029031; WO 2005009988; WO 2005009987; WO 2005012287; WO 2005030753; WO 2005030766; WO 2004103281; WO 2002072536; WO 2002090326; WO 2003053945; WO 2003068749; WO 2005016915; WO 2005016922; WO 2005063260; WO 2007042906; WO 2006122772; WO 2005105798; WO 2006105971; WO 2006122799; WO 2006122776; WO 2006122773; WO 2006122771; WO 2006122777; WO 2006122770; WO 2006136245; WO 2004069792; WO 2006058338; WO 2006102645; WO 2007109355; WO 2006006741; WO 2006006740; WO 2005014580; WO 2007090134; WO 2003097586; WO 2004046133; WO 2004099177; WO 2005028445; WO 2005049601; WO 2005049613; WO 2005051390; WO 2005080391; WO 2006100520; WO 2006120481; WO 2006038041; WO 2006122200; WO 2007010383; WO 2002008221; WO 2006007851; WO 2006029142; WO 2003062209; WO 2004055003; WO 2004055004; WO 2004056774; WO 2005007648; WO 2005007646; WO 2005007652; WO 2005009977; WO 2005009982; WO 2005009980; WO 2005023807; WO 2005087227; WO 2006026135; WO 2006042289; WO 2006071538; WO 2006078992; WO 2006081388; WO 2007047575; WO 2007047576; WO 2004033435; WO 2005121116; WO 2005120510; WO 2007065662; WO 2007065888; WO 2007065663; WO 2002076946; WO 2007054480; WO 2007054474; WO 2005095329; WO 2006016218; WO 2006051378; WO 2006095263; WO 2006097817; WO 2006103503; WO 2005123666; WO 2006045498; WO 2004058754; WO 2005032493; WO 2005066171; WO 2005046683; WO 2006093832; WO 2007100758; WO 2006024776; WO 2007010138; WO 2007010144; and WO 2007088277; the disclosure of each of which is incorporated herein by reference.

In some embodiments, determination of antinociceptive Effect by topical application of a TRPV1 antagonist can also be determined using the in vivo assays as disclosed herein in the Examples.

The amount of TRPV-1 antagonist in the composition may vary. According to certain embodiments, the amount of TRPV-1 antagonist is from about 0.05% to about 5%, such as from about 0.1% to about 2%, such as from about 0.2% to about 1%, such as from about 0.2% to about 0.5%, or 0.05%-0.1%, or 0.1-0.2%, or 0.2-0.4%, or 0.4-0.6%, or 0.6-0.8%, or 0.8-1.0%, or 1.0-2.0%, or 2.0-3.0%, or 3.0-4.0% or 4.0-5.0% by weight of the composition (w/v). In some embodiments, the amount of TRPV-1 antagonist is from about 1.0% to about 10%, e.g., any range such as from 1-2%, or 2-3%, or 3-4%, or 4-5%, or 5-6%, or 6-7%, or 7-8%, or 8-9%, or 9-10% by weight of the composition (w/v). In some embodiments, the amount of TRPV-1 antagonist is from about 5.0% to about 20.0%, e.g., any range such as from 5-6%, or 6-7%, or 7-8%, or 8-9%, or 9-10%, or 10-12%, or 12-14%, or 14-16%, or 16-18%, or 18-20% or 10-15%, or 15-20%, by weight of the composition (w/v).

The TRPV-1 antagonist administered according to the methods and compositions as disclosed herein comprise a pharmaceutically effective amount of one or more of the specified TRPV1 antagonists. As used herein, a “pharmaceutically effective amount” refers to that amount of one or more TRPV1 antagonists that prevents or alleviates acute injury or wound pain. In some embodiments, TRPV-1 antagonist are present in a composition for administration topically to the skin, or skin surrounding the wound or tissue injury, wherein the total amount of TRPV1 antagonist will be about 0.001 to 5.0% (w/v), or in the amount of TRPV1 antagonists is about 0.01 to about 5.0% (w/v). In some embodiments, a “physiologically relevant concentration” or “pharmaceutically effective amount” is less than 5.0% (w/v). In some embodiments, a physiologically relevant concentration is 0.001%-0.5% (w/v), or 0.01%-0.5%, or 0.1%-0.5%, or 0.5%-1.0%, or 1.0%-1.5%, or 1.5%-2.0%, or 2.0-3.0%, or 3.0%-4.0%, or 4.0%-5.0%. In some embodiments, the effective amount of a TRPV1 antagonist is between 5.0%-10.0%, e.g., any range such as from 5-6%, or 6-7%, or 7-8%, or 8-9%, or 9-10% by weight of the composition (w/v).

### Topical TRPV-1 Antagonists for Treatment of Tissue Injury and Wounds

As disclosed herein, the compositions and methods as disclosed herein relate to compositions comprising a TRPV1 antagonist for topical delivery to reduce the acute or nociceptive pain sensation in the subject with a tissue injury, wound or incision. The tissue injury for treatment with the compositions and devices disclosed herein can be any one or a combination of: a surgical incision, a non-surgical graze, skin abrasion, open wound, a deep wound, superficial wound, laceration, puncture, avulsion. In some embodiments, the tissue injury is a surgical incision. An abrasion occurs when the skin rubs or scrapes against a rough or hard surface (e.g., road rash). An incision is a result of a sharp object, such as a knife, shard of glass, or razor blade cutting into the tissue or skin. A deep incision can damage tendons, ligaments, and muscles. A laceration is a deep cut or tearing of the skin, and typically occur with accidents with knives, tools, and machinery. A puncture is a small hole caused by a long, pointy object, such as a nail, needle, or ice pick. Punctures can also be a result of a bullet. An avulsion is a partial or complete tearing away of skin and tissue, and can occur during violent accidents, such as body-crushing accidents, explosions, and gunshots.

A wound generically refers to a tissue injury caused by physical means. In everyday parlance, wounds typically refer to skin injuries.

In some embodiments, the wound or tissue injury is an acute wound or a chronic wound. In some embodiments, the wound or tissue injury is a non-penetrating wound, e.g., a wound which is a result of blunt trauma or friction with other surfaces; or where the wound does not break through the skin, and may include, Abrasions (scraping of the outer skin layer), lacerations (a tear-like wound), penetrating wounds (e.g., the result from trauma that breaks through the full thickness of skin; reaching down to the underlying tissue and organs, and includes, e.g., stab wounds (trauma from sharp objects, such as knives), skin cuts, surgical wounds (intentional cuts in the skin to perform surgical procedures), and gunshot wounds (wounds resulting from firearms).

In some embodiments, the wound or tissue injury is a burn. In alternative embodiments, the tissue injury is not a burn. In some embodiments, the compositions comprising a TRPV1 antagonist for topical administration, or devices as disclosed herein are not used in the treatment of a burn injury, e.g., a burn injury originating from a thermal exposure, (e.g., by exposure to a flame, a hot surface, a hot liquid, such as scalding-type burn injury, or exposure to extreme cold). In some embodiments, the compositions comprising a TRPV1 antagonist for topical administration, or devices as disclosed herein are not used in the treatment of a burn injury resulting from radiation exposure, for example, UVB exposure causing sunburn {see, e.g., U.S. Pat. Nos. 6,984,647 and 7,678,812), from contact with chemical agents, for example, exposure to alkaline material {see, e.g., Okada et al, “TRPV1 involvement in inflammatory tissue fibrosis in mice,” Amer. J. Pathology 178:2654-2664 (201 1)), or from friction {see, e.g., Pereira et al., “Development of animal model for studying deep second-degree thermal burns, ” J. Biomedicine Biotech. 2012: 1 -7 (2012)).

In some embodiments, the wound or tissue injury is not an eye injury. In some embodiments, the tissue injury is not ophthalmic, and the TRPV1 antagonist is not administered topically to the eye.

### Topical Delivery Systems

The topical administration of a TRPV1 antagonist as disclosed herein can be using any topical delivery device or system, which are commonly known to persons of ordinary skill. A “topical delivery device” any applicator or device that provides delivery of a compositions comprising one or more TRPV1 antagonists at or near the target site, e.g., at or in close proximity to a tissue injury, trauma or wound as needed for treatment of pain, inflammation or other disease or condition.

Aspects of the present invention relate to an applicator for topical delivery of a composition comprising a TRPV1 antagonist as disclosed herein. Applicators for topical delivery of a drug or compound are well known in the art and include, for example, but are not limited to, a wipe, a spray, pump or aerosol bottle, a dermal patch, a matrix, a brush, a dressing or sponge, a syringe applicator, optionally calibrated, an ampule, a bottle with irrigating nozzle, pipette dropper or liquid dispenser, a topical spreading applicator, a tube, a topical dosing device.

For example, the topical delivery applicator can be a bottle with a brush applicator, where the patient brushes or paints the composition comprising TRPV1 antagonist onto the tissue injury or wound target site. Alternatively, a topical delivery applicator can be a wipe or matrix comprising the TRPV1 antagonist, or where the person pours or soaks the wipe or matrix with the composition comprising a TRPV1 antagonist prior to applying it to the tissue injury or wound target site. Alternatively, a topical delivery applicator can be a bottle with a nozzle for dripping or spraying the composition comprising a TRPV1 antagonist to the tissue injury or wound target site, or pressed-air assisted spraying (e.g., pump action bottle) or aerosol spray for spraying the composition comprising a TRPV1 antagonist to the tissue injury.

In some embodiments, an applicator for topical delivery is a topical delivery system, such as, for example a topical spreading applicator, as disclosed in USD749225 or a topical dosing device as disclosed in US20050054991, which are incorporated herein in their entirety by reference.

In some embodiments, a TRPV1 antagonist is formulated for topical delivery as a powder, ointment or salve, aerosol, gel, emulsion, foam, cream or lotion.

In some embodiments, a TRPV1 antagonist is released locally from a topical delivery device at a dose of from about 0.3 mg/day or about 1.8 mg/day or about 3.6 mg/day to about 180 mg/day or about 360 mg/day. In some embodiments, a TRPV1 antagonist is released from a topical delivery device at a dose of 0.75 mg to 16 mg per day. In some embodiments, a TRPV1 antagonist can be released locally at a dose of from about 0.5 mg/day to about 15 mg/day.

In some embodiments, the initial burst or bolus release from the topical delivery device is about 2 to 20 times higher from 1 hour to about 3 hours than a sustained release daily dose released from the device. In some embodiments, a TRPV1 antagonist can have a burst release in 24 to 48 hours.

### Topical Compositions and Pharmaceutical Formulations Thereof

In some embodiments, a TRPV1 antagonist is formulated for topical delivery as a powder, ointment or salve, aerosol, gel, emulsion, foam, cream or lotion. Appropriate doses and formulations of a composition comprising a TRPV1 antagonist for topical application or for a transdermal administration can be determined by an ordinary skilled artisan according to known methods, such as those discussed in Hesselink, 2016 Topical Analgesics: Critical Issues Related to Formulation and Concentration. J Pain Relief 5:274, or Casale R, et al., Topical Treatments for Localized Neuropathic Pain. Curr Pain Headache Rep. 2017 Mar;21(3):15, which ares incorporated herein in their entirety by reference.

In some embodiments, TRPV1 antagonists for use in the compositions, kits, and methods as disclosed herein are for topical application and may include a cosmetically-acceptable topical carrier. A cosmetically-acceptable topical carrier may contain ingredients commonly used, such as water, monoalcohols (such as ethanol and isopropanol); glycols and polyols (such as glycerin, propylene glycol, propanediol, 1,4-butanediol, 1,3-butanediol, 1,2-butanediol, hydroxyethyl urea, sorbitol, sorbitan, xylitol and polyglycerols); glycerin, and combinations thereof. According to certain embodiments, a carrier can includes water.

The amount of cosmetically-acceptable topical carrier in the composition may range from about 30% to about 99%, such as from about 40% to about 95%, such as from about 50% to about 95%, such as from about 60% to about 90% by weight of the composition.

In some embodiments, the composition is in the form of a concentrate. As such, the composition may consist essentially of a TRPV-1 antagonist for topical application. For example, with the composition comprising at least one TRPV-1 antagonist and optionally up to about 20% of a diluent such as water, a monoalcohol, a glycol/polyol, or combinations thereof.

In some embodiments, the compositions as disclosed herein may include additional ingredients commonly used in topical compositions. Examples of additional ingredients include but are not limited to surfactants/emulsifiers (cationic, anionic, non-ionic, and zwitterionic), humectants, emollients and hydrophobic compounds, conditioning agents, opacifying agents, chelating agents, conditioning agents, additional preservatives, skin benefit agents, fragrances, water-soluble or dispersible polymers, and active ingredients (e.g., sunscreens, anti-aging actives, anti-acne actives, antibiotics, antimicrobial agents, and the like).

In some embodiments, the composition is aqueous and the pH of the composition is about 6.5 or greater, such as from about 6.5 to about 8.5, such as from about 7.5 to about 8.5.

Compositions of the present invention are particularly suitable for topically applying the TRPV1 antagonist to skin or mucosa. In particular embodiments, the composition is topically applied to, or within close proximity to a tissue injury or wound in the subject, and is not rinsed from the skin, wound or tissue injury. In some embodiments, topical administration of a composition comprising a TPRV1 antagonist as disclosed herein relates to applying or administering the composition comprising a TRPV1 antagonist to a specific area on, or in the body, for example, a composition comprising a TRPV1 antagonist is applied on the skin surrounding a wound, or in the wound, or at, or near a target site, e.g., at or in close proximity to a tissue injury, for example, on the exposed surface of the wound (e.g., where the skin is broken and/or the underlying tissues). In some embodiments, the composition comprising a TRPV1 antagonist may be contained within or be in fluid communication with an applicator that is suitable for dispensing it.

The composition comprising a TRPV1 antagonist as disclosed herein can take the form of solutions, suspensions, emulsions, pellets, multiparticulates, capsules, capsules containing liquids, powders, sustained-release formulations, aerosols, sprays, ointments, gels, salves, plasters, transdermal patches, suspensions, or any other topical form suitable for use. In one embodiment, the composition is in the form of a capsule (see e.g., U.S. Pat. No. 5,698,155). Other examples of suitable pharmaceutical excipients are described by Radebough et al, “Preformulation,” pp. 1447-1676 in Remington's Pharmaceutical Sciences Vol. 2 (Gennaro, ed., 19^{th} ed., Mack Publishing, Easton, Pa., 1995), incorporated herein by reference.

In some embodiments, a composition comprising a TPRV1 antagonist is applied in a transdermal formulation. In alternative embodiments, the TPRV1 antagonist is formulated for non-transdermal application. In such embodiments, a topical formulation of a composition comprising a TRPV1 antagonist is characterized by local analgesic effects in the absence of systemic effects, and does not require a transdermal delivery formulation. Dosages of a composition comprising a TRPV1 antagonist for topical administration or administration via a transdermal patch can be determined by one of ordinary skill in the art, for example, as discussed in Hesselink JMK (2016) Topical Analgesics: Critical Issues Related to Formulation and Concentration. J Pain Relief 5:274, which is incorporated herein in its entirety by reference.

Dosage forms for topical administration of a TRPV1 antagonist as disclosed herein powders, sprays, ointments and in some embodiments, inhalants. In some embodiments, a TRPV1 antagonist may be mixed under sterile conditions with a pharmaceutically acceptable carrier and any needed preservatives, buffers or propellants which may be required. In some embodiments, opthalmic formulations, eye ointments, powders and solutions are also contemplated as being within the scope of this invention for topical application of a TRPV1 antagonist to an eye wound, such as an incision to the eye. In some embodiments, topical administration of a TRPV1 antagonist as disclosed herein is not for administration to the eye. In some embodiments, if the subject is administered a TRPV1 antagonist to the eye according to the methods, compositions and devices disclosed herein, the subject has a wound to the eye (e.g., an accidental trauma, incision, abrasion, laceration, puncture or avulsion to the eye), and does not have dry eye syndrome, or inflammation of the eye, inflammation, dry eye syndrome,

In another embodiment, a TRPV1 antagonist as disclosed herein, or a pharmaceutically acceptable salt or solvate thereof can be topically administered in a vesicle, in particular a liposome (see Langer, “New Methods of Drug Delivery,” Science 249: 1527-1533 (1990); Lopez-Berestein, “Treatment of Systemic Fungal Infections with Liposomal-Amphotericin B,” Liposomes in the Therapy of Infectious Disease and Cancer, pp. 317-327 (1989); and Treat et al, “Liposome encapsulated doxorubicin—preliminary results of phase I and phase II trials” Liposomes in the Therapy of Infectious Disease and Cancer, pp. 353-365 (1989). TRPV1 antagonists as disclosed herein invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art. See, for example, Prescott, Ed., Methods in Cell Biology, Volume XIV, Academic Press, New York, N.Y. (1976), p. 33 et seq.

In some embodiments, a TRPV1 antagonist as disclosed herein, or a pharmaceutically acceptable salt or solvate thereof can be topically administered via a nanoparticle or microparticle, e.g., nanoparticles and microparticles formulated for skin drug delivery, as disclosed in Prow et at al, Nanoparticles and microparticles for skin drug delivery, Advanced Drug Delivery Reviews, 2011, 63(6), 470-491, which is incorporated herein in its entirety by reference.

In some embodiments, a TRPV1 antagonist as disclosed herein, or a pharmaceutically acceptable salt or solvate thereof is formulated for a low systemic uptake (i.e., poor lipid solubility) in order to reduce systemic side effects. In order to reduce the solubility of a TRPV1 antagonist as disclosed herein, or to assist in obtaining a controlled release effect, a TRPV1 antagonist can be utilized as the free base or utilized in a salt, which has relatively lower solubility. For example, the present application can utilize an insoluble salt such as a fatty acid salt. Representative fatty acid salts include salts of oleic acid or linoleic acid. In preferred embodiments fatty acid salts with between 8 to 20 carbons are used to produce salts with low solubility. Most preferably, fatty acid salts with between 12 to 18 carbons are used. Other embodiments can utilize a lipid soluble salt of a TRPV1 antagonist as disclosed herein.

In yet another embodiment, a TRPV1 antagonist as disclosed herein or a pharmaceutically acceptable salt or solvate thereof can be delivered in a controlled-release system or sustained-release system (see, e.g., Goodson, “Dental Applications,” pp. 1 15-138 in Medical Applications of Controlled Release, Vol. 2, Applications and Evaluation, Langer and Wise, eds., CRC Press (1984), hereafter “Goodson”). Other controlled- or sustained-release systems discussed in the review by Langer, Science 249: 1527-1533 (1990) can be used. In one embodiment, a pump can be used (Langer, Science 249:1527-1533 (1990); Sefton, “Implantable Pumps,” in CRC Crit. Rev. Biomed. Eng. 1_4(3):201 -240 (1987); Buchwald et al, “Long-term, Continuous Intravenous Heparin Administration by an Implantable Infusion Pump in Ambulatory Patients with Recurrent Venous Thrombosis,” Surgery 88:507-516 (1980); and Saudek et al., “A Preliminary Trial of the Programmable Implantable Medication System for Insulin Delivery,” New Engl. J. Med. 321 :574-579 (1989)). In another embodiment, polymeric materials can be used (see Goodson; Smolen et al, “Drug Product Design and Performance,” Controlled Drug Bioavailability Vol. 1, John Wiley & Sons, New York (1984); Langer et al, “Chemical and Physical Structure of Polymers as Carriers for Controlled Release of Bioactive Agents: A Review,” J. Macromol. Sci. Rev. Macromol. Chem. C23(1):61-126 (1983); Levy et al, “Inhibition of Calcification of Bioprosthetic Heart Valves by Local Controlled-Release Diphosphonate,” Science 228: 190-192 (1985); During et al, “Controlled Release of Dopamine from a Polymeric Brain Implant: In Vivo Characterization,” Ann. Neurol. 25:351-356 (1989); and Howard et al, “Intracerebral drug delivery in rats with lesion-induced memory deficits,” J. Neurosurg. 71: 105 (1989)). In yet another embodiment, a controlled- or sustained-release system comprising a TRPV1 antagonist as disclosed herein can be placed in proximity of the tissue injury or wound, thus requiring only a fraction of the systemic dose.

A TRPV1 antagonist as disclosed herein can optionally comprise a suitable amount of a pharmaceutically acceptable excipient so as to provide the form for proper topical administration to the animal. Such a pharmaceutical excipient can be a diluent, suspending agent, solubilizer, binder, disintegrant, preservative, coloring agent, lubricant, and the like. The pharmaceutical excipient can be a liquid, such as water or an oil, including those of petroleum, animal, vegetable, or synthetic origin, such as peanut oil, soybean oil, mineral oil, sesame oil, and the like. In some embodiments, a pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to a subject or animal.

For topical application, saline solutions and aqueous dextrose and glycerol solutions can also be employed as liquid excipients. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The compositions, if desired, can also contain wetting or emulsifying agents, or pH buffering agents.

In one embodiment, compositions comprising a TRPV1 antagonist as disclosed herein for topical administration comprise sterile isotonic aqueous buffer. Where necessary, the compositions can also include a solubilizing agent. A TRPV1 antagonist as disclosed herein or a pharmaceutically acceptable salt or solvate thereof for topical administration can optionally include a local anesthetic such as benzocaine or prilocaine.

Generally, a TRPV1 antagonist as disclosed herein and other ingredients (e.g., pharmaceutically acceptable carrier or second therapeutic agent) are supplied either separately or mixed together in unit dosage form, for example, as a dry lyophilized powder or water free concentrate in a hermetically sealed container such as an ampule or sachette indicating the quantity of active agent. For topical administration of TRPV1 antagonist as disclosed herein or a pharmaceutically acceptable salt or solvate thereof, it can be dispensed, for example, with an infusion bottle containing sterile pharmaceutical grade water or saline. In some embodiments, a TRPV1 antagonist as disclosed herein or a pharmaceutically acceptable salt or solvate thereof can be present in an ampule, and optionally a separate ampule can be provided comprising sterile water or saline, so that the ingredients can be mixed prior to topical administration to the subject.

In some embodiments, a TRPV1 antagonist as disclosed herein and a pharmaceutically acceptable salt and solvate thereof can be administered by controlled-release or sustained-release means or by delivery devices that are known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled- or sustained-release of one or more active ingredients using, for example, hydropropylmethyl cellulose, ethylcellulose, other polymer matrices, gels, permeable membranes, osmoptic systems, multilayer coatings, microparticles, liposomes, microspheres, or a combination thereof to provide the desired release profile in varying proportions. Suitable controlled- or sustained-release formulations known to those of ordinary skill in the art, including those described herein, can be readily selected for use with the TRPV1 antagonists as disclosed herein. Therefore, also encompassed herein are single unit dosage forms of a TRPV1 antagonist for topical administration such as, but not limited to, dermal patches, matrix, dressing, sponge, dermal patch, salve, cream, lotion, foam, emulsion, ointment, powder or gel that are adapted for controlled- or sustained-release.

Controlled- or sustained-release pharmaceutical compositions can have a common goal of improving drug therapy over that achieved by their non-controlled or non-sustained release counterparts. In one embodiment, a controlled- or sustained-release composition comprises a minimal amount of a TRPV1 antagonist as disclosed herein or a pharmaceutically acceptable salt or solvate thereof to cure or control the condition in a minimum amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of a TRPV1 antagonist as disclosed herein and can thus reduce the occurrence of adverse side effects.

Controlled- or sustained-release compositions can be designed to immediately release an amount of a TRPV1 antagonist as disclosed herein or a pharmaceutically acceptable salt or solvate thereof that promptly produces the desired therapeutic or prophylactic effect, and gradually and continually release other amounts of a TRPV1 antagonist as disclosed herein, or a second therapeutic agent or a pharmaceutically acceptable salt or solvate thereof to maintain this level of therapeutic or prophylactic effect over an extended period of time. To maintain a constant level of a TRPV1 antagonist as disclosed herein at the site of the tissue injury or wound, the TRPV1 antagonist or a pharmaceutically acceptable salt or solvate thereof can be released from the dosage form at a rate that will replace the amount of TRPV1 antagonist being metabolized or broken down or excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of a TRPV1 antagonist as disclosed herein or a pharmaceutically acceptable salt or solvate thereof that is topically administered and is effective in the treatment of pain at the site of tissue injury or wound can be determined by standard clinical techniques. In addition, in vitro or in vivo assays can optionally be employed to help identify optimal dosage ranges. The precise dose to be employed will also depend on which TRPV1 antagonist is being used, the formulation and topical application method (e.g., aerosol, dermal patch, wipe, powder, ointment, cream, lotion etc.) and the seriousness of the tissue injury or wound and can be decided according to the judgment of a practitioner and/or each person or animal's circumstances.

Suitable effective dosage amounts, however, will, in one embodiment, range from about 0.01 mg/kg of body weight to about 2500 mg/kg of body weight. In another embodiment, effective dosage amounts will be about 100 mg/kg of body weight or less. In one embodiment, the effective dosage amount ranges from about 0.01 mg/kg of body weight to about 100 mg/kg of body weight of a compound of formula (I) or a pharmaceutically acceptable salt or solvate thereof; in another embodiment, about 0.02 mg/kg of body weight to about 50 mg/kg of body weight; and in another embodiment, about 0.025 mg/kg of body weight to about 20 mg/kg of body weight.

In one embodiment, an effective dosage amount of a TRPV1 antagonist as disclosed herein is topically administered as soon as possible after tissue injury or wound to the subject, e.g., within about 0 and 3 hours of the injury, or between 0 and 1 hour of the injury, where 0 is the time of the occurrence of the injury. In some embodiments, a TRPV1 antagonist as disclosed herein is topically administered about every 30 minutes, or about every 1 hour, or about every 2 hours, or about 3 hours, or about every 4-6 hours, or about every 6-12 hours, or about every 12-24 hours to the site of the tissue injury or wound until pain is abated.

The effective dosage amounts described herein refer to total amounts administered; that is, if more than one TRPV1 antagonist as disclosed herein is administered, the effective dosage amounts correspond to the total amount administered.

In some embodiments, a composition comprising one or more TRPV1 antagonists as disclosed herein are topically administered in an effective amount, e.g., a therapeutically effective amount.

The phrase “therapeutically effective amount” of topical administration of a TRPV1 antagonist as disclosed herein means a sufficient amount of the TRPV1 antagonist to treat disorders, at a reasonable benefit/risk ratio applicable to any medical treatment. It will be understood, however, that the total daily usage of a TRPV1 antagonists will be decided by the person administrating the composition comprising the TRPV1 antagonists, or an attending physician within the scope of sound medical judgment. The specific therapeutically effective dose level for topical application for any particular patient or subject will depend upon a variety of factors including the extent and type of the tissue injury or wound or pain disorder being treated, and the severity of the tissue injury, wound or pain disorder; activity of the specific TRPV1 antagonist employed; the specific topical formulation and delivery of the TRPV1 antagonists employed; the age, body weight, general health, sex and diet of the subject or patient; the time of administration, topical administration method, and rate of excretion of the specific TRPV1 antagonists employed; the duration of the treatment; drugs used in combination or coincidental with the specific TRPV1 antagonists employed; and like factors well-known in the medical arts. For example, it is well within the skill of the art to start topical administration of a TRPV1 antagonists at dose levels lower than required to achieve the desired therapeutic effect and to gradually increase the dosage until the desired effect is achieved.

Where a cell capable of expressing TRPV1 is contacted with a TRPV1 antagonist in vitro, the amount effective for inhibiting the TRPV1 receptor function in a cell will range from about 0.01 μg/L to about 5 mg/L; in one embodiment, from about 0.01 μg/L to about 2.5 mg/L; in another embodiment, from about 0.01 μg/L to about 0.5 mg/L; and in another embodiment, from about 0.01 μg/L to about 0.25 mg/L, of a solution or suspension of a pharmaceutically acceptable carrier or excipient. In one embodiment, the volume of solution or suspension comprising the compound of formula (I) or a pharmaceutically acceptable salt or solvate thereof, is from about 0.01 μl., to about 1 mL. In another embodiment, the volume of solution or suspension is about 200 μL.

In some embodiments, compositions as disclosed herein comprising a TRPV1 antagonist may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the topical formulation of a TPRV1 antagonist can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a topically administered drug form is accomplished by dissolving or suspending the drug in an oil vehicle.

A TRPV1 antagonist as disclosed herein, and the pharmaceutically acceptable salts and solvates thereof, can be assayed in vitro or in vivo for the desired therapeutic or prophylactic activity prior to use in humans. Animal model systems can be used to demonstrate safety and efficacy.

In some embodiments, the composition comprises a TRPV1 antagonist in an amount of from about 0.1% to about 75% by weight (w/v).

### Topical Application of a TRPV1 Antagonist With a Second Therapeutic Agent

In some embodiments, a TRPV1 antagonist as disclosed herein is topically administered alone, or in combination with one or more other TRPV1 antagonist as disclosed herein, or in combination (i.e. co-administered) with one or more additional pharmaceutical agents (i.e., a second therapeutic agent). In some embodiments, the second therapeutic agent is a pain management medication as that term is described herein. Accordingly, the disclosure herein relates to methods for inhibiting TRPV1 function in a cell capable of expressing TRPV1 can further comprise contacting the cell with an effective amount of a second therapeutic agent, as disclosed herein, including but not limited to a TRPA1 antagonist.

In some embodiments, the effective amount of the second therapeutic agent when used with a TRPV1 antagonist is less than the effective amount of the second therapeutic agent when used alone (or not in the presence or in combination with a TRPV1 antagonist). In some embodiments, a TRPV1 antagonist and second therapeutic agent are synergistic, in that they work together such that one agent increases the effectiveness of the other. For example, the topical application of a TRPV1 antagonist increases the effectiveness of the topical application of the secondary therapeutic agent, such that the effective dose of a topically applied secondary therapeutic agent is lower in the presence of the TRPV1 antagonist, (and higher in the absence of the TRPV1 antagonist). In some embodiments, the combined analgesic effect of a topically applied TRPV1 antagonist and topically applied second therapeutic agent is greater than when each of these agents are used alone. Synergistic effects are typically detected when the TRPV1 antagonist and second therapeutic agent work by different mechanisms so together they form a stronger analgesic effect than then they are used individually. In some embodiments, a TRPV1 antagonist and second therapeutic agent are additive, in that combined analgesic effect of a topically applied TRPV1 antagonist and topically applied second therapeutic agent is equal to the sum of the analgesic effect when these two agents are used alone. Additive effects are typically detected when the TRPV1 antagonist and second therapeutic agent work by the same or similar mechanism.

In some embodiments, a TRPV1 antagonist as disclosed herein or a pharmaceutically acceptable salt or solvate thereof, may be administered topically, in combination with one or more non-steroidal anti-inflammatory drug (NSAID) such as, but not limited to, aspirin, acetaminophen, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin and zomepirac.

Examples of a second therapeutic agents useful in the compositions, methods, kits and devised as disclosed herein also include, but are not limited to an anti-inflammatory agent, an analgesic agent, or an osteoinductive growth factor or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine [2-hydroxy-5-[-4-[C2-pyridinylamino)sulfonyl]azo]benzoic acid, sulindac, tepoxalin or tolmetin; as well as antioxidants, such as dithiocarbamate, steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone, lidocane, ketamine, muscle relaxants such as baclofen, ibuprofen, aspirin, diethylether, indomethacin, COX-2 inhibitors (e.g., diclofenac), tricyclic antidepresent angents (e.g., doxepin) or a combination thereof.

In some embodiments, additional suitable analgesic agents useful in the methods, compositions, kits and devices as disclosed herein as a second therapeutic agent to be applied topically with a topically applied TRPV 1 antagonist includes, but are not limited to, acetaminophen, bupivacaine, lidocaine, gamma-aminobutyric acid (GABA), clonidine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, α2-adrenergic receptors (e.g., amitriptyline, clonidine (also acts as an imidazonline-receptor agonist), Ambroxol, or a combination thereof.

In some embodiments, a second therapeutic agent includes any one or more of ketamine, amitriptyline or baclofen. In some embodiments, if ketamine is used as the second therapeutic agent to be administered topically with a topically applied TRPV1 antagonist, it is used at between 1%-10% w/v. In some embodiments, the dose of ketamine is less than the dose of ketamine that is needed for analgesic effect if ketamine is used alone, and the dose can be, for example, less than 10%, or less than 9%, or less than 8%, or less than 7%, or less than 6%, or less than 5%, or less than 4%, or less than 3%, or less than 2%, or less than 1% but more than 0.001%.

In some embodiments, if amitriptyline is used as the second therapeutic agent to be administered topically with a topically applied TRPV1 antagonist, it is used at between 1%-10% w/v. In some embodiments, the dose of amitriptylin is less than the dose of amitriptylin that is needed for analgesic effect if amitriptylin is used alone, and the amount of amitriptylin as a second therapeutic agent can be, for example, more than 1% but less than 10%, or less than 9%, or less than 8%, or less than 7%, or less than 6%, or less than 5%, or less than 4%, or less than 3%, or less than 2%, or less than 1% but more than 0.001%.

In some embodiments, if baclofen is used as the second therapeutic agent to be administered topically with a topically applied TRPV1 antagonist, it is used at between 0.05%-5.0% w/v. In some embodiments, the dose of baclofen is less than the dose of baclofen that is needed for analgesic effect if baclofen is used alone, and the amount of baclofen as a second therapeutic agent can be, for example, less than less than 5% or less than 4% or less than 3%, or less than 2%, or less than 1%, or less than 0.5%, or less than 0.25%, or less than 0.2%, or less than 0.1% or less than 0.05%, but more than 0.001%.

In some embodiments, if lidocane is used as the second therapeutic agent, it is used at between 0.001.0-5.0% w/v. In some embodiments, the dose of lidocane is less than the dose of topical lidocane that is needed for analgesic effect if lidocane is used alone, and the amount of lidocane as a second therapeutic agent can be, for example, less than 5%, or less than 4%, or less than 3%, or less than 2%, or less than 1%, or less than 0.5%, or less than 0.25%, or less than 0.1%, or less than 0.05%, or less than 0.001%, but more than 0.0005%.

In some embodiments, if diclofenac is used as the second therapeutic agent to be administered topically with a topically applied TRPV1 antagonist, it is used at between 0.001%.-2.0% w/v. In some embodiments, the dose of diclofenac is less than the dose of topical diclofenac that is needed for analgesic effect if diclofenac is used alone, and the amount of diclofenac as a second therapeutic agent can be, for example, less than 2%, or less than 1.5% or less than 1%, or less than 0.5%, or less than 0.25%, or less than 0.1%, or less than 0.05%, or less than 0.001%.

In some embodiments, if doxepepin is used as the second therapeutic agent to be administered topically with a topically applied TRPV1 antagonist, it is used at between 0.001%.-5.0% w/v. In some embodiments, the dose of diclofenac is less than the dose of topical diclofenac that is needed for analgesic effect if diclofenac is used alone, and the amount of diclofenac as a second therapeutic agent can be, for example, less than 5%, or less than 4%, or less than 3%, or less than 2%, or less than 1%, or less than 0.5%, or less than 0.25%, or less than 0.1%, or less than 0.05%, or less than 0.001%, but more than 0.0005%.

In some embodiments, if clonidine is used as the second therapeutic agent to be administered topically with a topically applied TRPV1 antagonist, it is used at between 0.001%.-0.1% w/v. In some embodiments, the dose of clonidine c is less than the dose of topical clonidine that is needed for analgesic effect if clonidine is used alone, and the amount of clonidine as a second therapeutic agent can be, for example less than 0.1%, or less than 0.09%, or less than 0.08%, or less than 0.07%, or less than 0.06%, or less than 0.05%, or less than 0.04%, or less than 0.03%, or less than 0.02%, or less than 0.01%, or less than 0.005% or less than 0.001%, but more than 0.0005%.

In some embodiments, if ambroxol is used as the second therapeutic agent to be administered topically with a topically applied TRPV1 antagonist, it is used at between 2%-20% w/v. In some embodiments, the dose of ambroxol is less than the dose of ambroxol that is needed for analgesic effect if ambroxol is used alone, and the amount of ambroxol as a second therapeutic agent can be, for example, more than 2% but less than 20%, or less than 18%, or less than 16%, or less than 15%, or less than 14%, or less than 12%, or less than 10%, or less than 9%, or less than 8%, or less than 7%, or less than 6%, or less than 5%, or less than 4%, or less than 3%, or less than 2%, or less than 1% but more than 2.0%.

In some embodiments, a second therapeutic agent is an osteogenic protein. Exemplary osteogenic proteins include, but are not limited to, OP-1, OP-2, OP-3, BMP-2, BMP-3, BMP-3b, BMP-4, BMP-5, BMP-6, BMP-9, BMP-10, BMP-11, BMP-12, BMP-13, BMP-14, BMP-15, GDF-1, GDF-2, GDF-3, GDF-5, GDF-6, GDF-7, GDF-8, GDF-9, GDF-10, GDF-11, GDF-12, CDMP-1, CDMP-2, CDMP-3, DPP, Vg-1, Vgr-1, 60A protein, NODAL, UNIVIN, SCREW, ADMP, NEURAL, and TGF-beta. As used herein, the terms “morphogen,” “bone morphogen,” “BMP,” “osteogenic protein” and “osteogenic factor” embrace the class of proteins typified by human osteogenic protein 1 (hOP-1).

Combination therapy includes administration of a single pharmaceutical dosage formulation containing one or more TRPV1 antagonists as disclosed herein and one or more additional pharmaceutical agents, as well as administration of a TRPV1 antagonist as disclosed herein and each additional pharmaceutical agent, in its own separate pharmaceutical dosage formulation. For example, a TRPV1 antagonist as disclosed herein and one or more additional pharmaceutical agents, may be administered to the patient together, in a single topical dosage composition having a fixed ratio of each active ingredient, such as an ointment, lotion, gel, spray, aerosol etc. or each agent may be administered in separate topical dosage formulations.

The phrase “pain management medication” includes one or more therapeutic agents that are administered to prevent, alleviate or remove pain entirely. These include anti-inflammatory agents, muscle relaxants, analgesics, anesthetics, narcotics, and so forth, and combinations thereof.

Accordingly, in some embodiments, one or more TRPV1 antagonists are administered in a therapeutically effective amount with a second therapeutic agent. In some embodiments, a second therapeutic agent is a TRPA1 antagonist. Any TRPA1 antagonist known to one of ordinary skill in the art can be used. Exemplary TRPA1 antagonists are disclosed in US Application 2015/0105406, and include alcohols (WO2013103155), amino ketones (WO2012050512 and Bioorg. Med. Chem. Lett. 2012, 22, 5485), decalins (WO 201 1043954), oximes (WO2009089082, WO2009089083 and Bioorg. Med. Chem. Lett. 2010, 20, 276), prolines and aminoacid derivatives (WO2010141805, EP2520566, WO2013108857 and WO2014049047), pyrimidinedione /xanthines based compounds (WO2007073505, WO2009002933, WO20091 18596, WO2009144548, WO2009158719, WO2010004390, WO2010036821 , WO2010075353, WO2010109287, WO2010109328, WO02010109329, WO2010109334, WO2010125469, WO2010132838, WO2010138879, WO201 1 1 14184, WO201 1 132017, WO2012176105, WO2012085662, WO2013023102 and Med. Chem. Comm. 2012, 3, 187), thioureas (WO2007073505, WO2009147079 and Bioorg. Med. Chem. Lett 2012, 22, 797), and various other structures (such as in WO2007098252 and WO2012152940). International publications WO2016042501 WO2015155306, WO2010109287, WO2010109328, WO2010109329, WO2010109334, WO2009118596, WO2009144548, WO2010004390, IN200802512, WO2010125469, WO2011114184, WO2011132017, WO2007073505, WO2010075353, WO2009158719, WO2009002933, WO2012085662 and WO2010132838 disclose various 2-amino-4-arylthiazole compounds which are useful for treating disorders related to TRPA1, as well as 2-Amino-4-arylthiazole compounds, and other TRPA1 antagonist compounds reviewed in Preti et al., Pharmaceutical Patent Analyst, 2015, 4(2); 75-94Transient receptor potential ankyrin 1 (TRPA1) antagonists. Accordingly, the disclosure herein relates to methods for inhibiting TRPV1 function in a cell capable of expressing TRPV1 can further comprise contacting the cell with an effective amount of a second therapeutic agent, as disclosed herein, including but not limited to a TRPA1 antagonist.

An effective amount of the second therapeutic agent(s) will be known to those skilled in the art depending on the agent. However, it is well within the skilled artisan's purview to determine the second therapeutic agent's optimal effective-amount range. A TRPV1 antagonist as disclosed herein or a pharmaceutically acceptable salt or solvate thereof, and the second therapeutic agent combined can act either additively or synergistically to treat a tissue injury or wound, or they can act independently of each other such that the TRPV1 antagonist as disclosed herein or pharmaceutically acceptable salt or solvate thereof treats the pain associated with the tissue injury or wound and the second therapeutic agent treats or prevents a second disorder, which can be the same as the tissue injury or wound or another disorder.

In one embodiment of the disclosure, where a second therapeutic agent is administered to an animal for treatment of a pain, the minimal effective amount of a TRPV1 antagonist as disclosed herein or pharmaceutically acceptable salt or solvate thereof will be less than its minimal effective amount would be where the second therapeutic agent is not administered. In this embodiment, a TRPV1 antagonist as disclosed herein or pharmaceutically acceptable salt or solvate thereof and the second therapeutic agent can be administered topically and act synergistically to treat the pain associated with a tissue injury or wound. In one embodiment, a TRPV1 antagonist as disclosed herein or pharmaceutically acceptable salt or solvate thereof is administered concurrently with a second therapeutic agent as a single composition comprising an effective amount of a TRPV1 antagonist as disclosed herein or pharmaceutically acceptable salt or solvate thereof for topical administration and an effective amount of the second therapeutic agent. Alternatively, a composition comprising an effective amount of a TRPV1 antagonist as disclosed herein or pharmaceutically acceptable salt or solvate thereof and a second composition comprising an effective amount of the second therapeutic agent are concurrently topically administered. In another embodiment, an effective amount of a TRPV1 antagonist as disclosed herein or pharmaceutically acceptable salt or solvate thereof is administered prior or subsequent to administration of an effective amount of the second therapeutic agent. In this embodiment, a TRPV1 antagonist as disclosed herein or pharmaceutically acceptable salt or solvate thereof is administered while the second therapeutic agent exerts its therapeutic effect, or the second therapeutic agent is administered while the TRPV1 antagonist as disclosed herein exerts its therapeutic effect for treating a Pain Condition.

In some embodiments, a second therapeutic agent as disclosed herein can be, but is not limited to, an opioid agonist, a non-opioid analgesic, a non-steroidal anti-inflammatory agent, an antimigraine agent, a Cox-II inhibitor, an antiemetic, a β-adrenergic blocker, an anticonvulsant, an antidepressant, a Ca²⁺-channel blocker, an anticancer agent, an agent for treating or preventing UI, an agent for treating or preventing an ulcer, an agent for treating or preventing IBD, an agent for treating or preventing IBS, an agent for treating addictive disorder, an agent for treating Parkinson's disease and parkinsonism, an agent for treating anxiety, an agent for treating epilepsy, an agent for treating a stroke, an agent for treating a seizure, an agent for treating a pruritic condition, an agent for treating psychosis, an agent for treating Huntington's chorea, an agent for treating ALS, an agent for treating a cognitive disorder, an agent for treating a migraine, an agent for treating vomiting, an agent for treating dyskinesia, an agent for treating depression, or any mixture thereof.

Examples of useful opioid agonists include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, noipipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, or any mixture thereof.

In certain embodiments, the opioid agonist is codeine, hydromorphone, hydrocodone, oxycodone, dihydrocodeine, dihydromorphine, morphine, tramadol, oxymorphone, or any mixture thereof.

Examples of useful non-opioid analgesics include, but are not limited to, non-steroidal anti-inflammatory agents, such as aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, or any mixture thereof. Other suitable non-opioid analgesics include the following, non-limiting, chemical classes of analgesic, antipyretic, nonsteroidal anti-inflammatory drugs: salicylic acid derivatives, including aspirin, sodium salicylate, choline magnesium trisalicylate, salsalate, diflunisal, salicylsalicylic acid, sulfasalazine, and olsalazin; para-aminophenol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid and meclofenamic acid; enolic acids, including oxicams (piroxicam, tenoxicam), and pyrazolidinediones (phenylbutazone, oxyphenthartazone); alkanones, including nabumetone; or any mixture thereof. For a more detailed description of the NSAIDs, see Insel, “Analgesic-Antipyretic and Anti-inflammatory Agents and Drugs Employed in the Treatment of Gout,” pp. 617-657 in Goodman & Gilman's The Pharmacological Basis of Therapeutics (Goodman et al, Eds., 9^{th} Ed., McGraw-Hill, New York 1996), and Hanson, “Analgesic, Antipyretic and Anti-Inflammatory Drugs,” pp. 1196-1221 in Remington: The Science and Practice of Pharmacy Vol 2 (Gennaro, ed., 19^{th} ed., Mack Publishing, Easton, Pa., 1 95), which are hereby incorporated by reference in their entireties.

The second therapeutic agent can also be an agent useful for reducing any potential side effects of a TRPV1 antagonist as disclosed herein. For example, the second therapeutic agent can be an antiemetic agent. Examples of useful antiemetic agents include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, or any mixture thereof.

Examples of useful Ca-channel blockers include, but are not limited to, bepridil, clentiazem, diltiazem, fendiline, gallopamil, mibefradil, prenylamine, semotiadil, terodiline, verapamil, amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, perhexiline, or any mixture thereof. Examples of useful therapeutic agents for treating or preventing anxiety include, but are not limited to, benzodiazepines, such as alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, and triazolam; non-benzodiazepine agents, such as buspirone, gepirone, ipsapirone, tiospirone, zolpicone, Zolpidem, and zaleplon; tranquilizers, such as barbituates, e.g., amobarbital, aprobarbital, butabarbital, butalbital, mephobarbital, methohexital, pentobarbital, phenobarbital, secobarbital, and thiopental; propanediol carbamates, such as meprobamate and tybamate; or any mixture thereof. Examples of useful therapeutic agents for treating or preventing a pruritic condition include, but are not limited to, naltrexone; nalmefene; danazol; tricyclics such as amitriptyline, imipramine, and doxepin; antidepressants such as those given below, menthol; camphor; phenol; pramoxine; capsaicin; tar; steroids; antihistamines; or any mixture thereof. Examples of useful therapeutic agents for treating or preventing vomiting include, but are not limited to, 5-HT₃ receptor antagonists such as ondansetron, dolasetron, granisetron, and tropisetron; dopamine receptor antagonists such as prochlorperazine, thiethylperazine, chlorpromazin, metoclopramide, and domperidone; glucocorticoids such as dexamethasone; benzodiazepines such as lorazepam and alprazolam; or any mixture thereof. Examples of useful therapeutic agents for treating or preventing depression include, but are not limited to, tricyclic antidepressants such as amitryptyline, amoxapine, bupropion, clomipramine, desipramine, doxepin, imipramine, maprotilinr, nefazadone, nortriptyline, protriptyline, trazodone, trimipramine, and venlaflaxine; selective serotonin reuptake inhibitors such as citalopram, (S)-citalopram, fluoxetine, fluvoxamine, paroxetine, and setraline; monoamine oxidase inhibitors such as isocarboxazid, pargyline, phenelzine, and tranylcypromine; psychostimulants such as dextroamphetamine and methylphenidate; or any mixture thereof.

A composition of the disclosure is prepared by a method comprising admixing a TRPV1 antagonist as disclosed herein or a pharmaceutically acceptable salt or solvate thereof with a pharmaceutically acceptable carrier or excipient. Admixing can be accomplished using methods known for admixing a compound and a pharmaceutically acceptable carrier or excipient. In one embodiment, a TRPV1 antagonist as disclosed or pharmaceutically acceptable salt or solvate thereof is present in the composition in an effective amount.

### Uses

In particular embodiments, the composition is topically applied to, or within close proximity to a tissue injury or wound in the subject, and is not rinsed from the skin, wound or tissue injury. In some embodiments, the TRPV1 antagonists as disclosed herein are topically administered, e.g., applied locally to the area of pain in the subject, e.g., applied on, or near the area of tissue injury or pain. In some embodiments, the TRPV1 antagonists as disclosed herein are topically administered, e.g., applied to focal sites of the sensation of pain in a subject.

In some embodiments, a TRPV1 antagonist as disclosed herein are administered locally to a site of tissue injury, trauma or wound, in that the TRPV1 antagonist is deposited on, or in close proximity to the site of injury, e.g., within about 0.1 cm, or preferably within about 10 cm, for example of the site of tissue injury, trauma or wound on the subject. For example, a dose of TRPV1 antagonist as disclosed herein is delivered locally, for example, 10%, 20%, 30%, 40%, 50%, 60%, 70%, 80%, 90%, 95%, 99%, or 99.9% less than the oral dosage or injectable dose. In turn, systemic side effects, such as for example, hyperthermia are minimized.

In some embodiments, a composition comprising a TPPV1 antagonist is topically administered to a subject, e.g., a mammalian subject, e.g., a human, for treating a disorder that may be ameliorated by inhibiting vanilloid receptor subtype 1 (TRPV1) receptor in need of such treatment. The method comprises topically administering a therapeutically effective amount of a TRPV1 antagonist as disclosed herein, in a method for treating pain in a subject, e.g., a mammal in need of such treatment, e.g., where the subject, e.g., human or other mammalian subject has a wound or tissue injury.

In some embodiments, the method comprises topically administering a therapeutically effective amount of a TRPV1 antagonist as disclosed herein, to a subject, e.g., a mammal or human subject in need of such treatment, for example, where the subject has nociception and/or the sensation of pain, post-surgical pain, including but not limited to allodynia (i.e., pain due to a stimulus that does not normally provoke pain), and hyperalgesia (i.e., increased response to a stimulus that is normally painful), thermal or mechanical (tactile) hyperalgesia. In some embodiments, the post-surgical pain includes mechanically-induced pain or resting pain. In other embodiments, the post-surgical pain includes resting pain. The pain can be primary (e.g., resulting directly from the pain-causing event) or secondary pain (e.g., pain associated with, but not directly resulting, from the pain-causing event).

In some embodiments, the method comprises topically administering a therapeutically effective amount of a TRPV1 antagonist as disclosed herein, to a subject whom has pain characterized by any one or more of thermal sensitivity, mechanical sensitivity and/or resting pain (e.g. persistent pain in the absence of external stimuli).

In some embodiments, the method comprises topically administering a therapeutically effective amount of a TRPV1 antagonist as disclosed herein to a subject to treatment acute or nociceptive pain, and results in a reduction, diminishment and/or minimization of the sensation of pain associated with surgery, incision, trauma or wound (including reduced, diminished, and/or minimized subjective perception of pain). In yet another aspect, the method comprises topically administering a therapeutically effective amount of a TRPV1 antagonist as disclosed herein to a subject to enhance recovery from surgery, as well as enhancing recovery from wound, traumatic injury, and/or incision.

In some embodiments, the method comprises topically administering a therapeutically effective amount of a TRPV1 antagonist as disclosed herein to a subject for treating post-surgical pain. The term “Post-surgical pain” (interchangeably termed “post-incisional” or “post-traumatic pain”) refers to pain arising or resulting from an external trauma such as a cut, puncture, incision, tear, or wound into tissue of an individual (including those that arise from all surgical procedures, whether invasive or non-invasive). As used herein, “post-surgical pain” does not include pain that occurs without an external physical trauma. In some embodiments, post-surgical pain is external pain, and topical application of the TRPV 1 antagonist is applied on, or near the area of the wound, cut, trauma, tear or incision made during surgery intentionally or deliberately (i.e., a surgical incision, or injury as part of the surgical procedure), or may occur accidentally (as with a traumatic wound).

In some embodiments, the TRPV 1 antagonists as disclosed herein are topically administered, e.g., applied locally to the area where pain is expected to occur in the subject, e.g., applied on, or near the area where tissue injury or pain may occur. For example, but by no way a limitation, a TRPV 1 antagonist as disclosed herein, can be applied topically, e.g., in a cream, lotion, gel, wipe, spray or aerosol, or by any of the administration methods as disclosed herein, to an area prior to an injection or catheter or i.v. line placement. For example, a TRPV 1 antagonist as disclosed herein can be administered topically to an area where an injection is to be administered (e.g., arm, leg, buttock, back, stomach, gum, etc.). In some embodiments, a TRPV 1 antagonist is applied topically an area where an injection or intravenous line (including epidural catheter placement) is to be administered, typically between 0-10 minutes, or about 30 sec, or about 1 min, or about 2 mins, or about 2-5 mins, or about 5-10 mins, or about 10-20 mins prior to the injection or i.v. line placement. Accordingly, the methods, compositions and kits are useful for topically administering TRPV 1 antagonists to the skin prior to an injection, blood being drawn, intravenous line placement or removal (e.g., i.v. drips, epidural catheters and other cannulas or catheters etc.) and the like. In some embodiments, the methods, compositions and kits are useful for topically administering TRPV 1 antagonists to the skin prior to removal of a foreign body, e.g., removal of a splinter, i.v., line, catheter and the like, in the subject. In some embodiments, such a subject is human, and can be an adult, teen, child, baby or neonate.

In some embodiments, the method comprises topically administering a therapeutically effective amount of a TRPV 1 antagonist as disclosed herein to a subject during field surgery, or combat casualty care, or combat anesthesia. It is appreciated that although reference is generally made herein to treating or preventing acute or nociceptive pain, including surgical incisions and post-surgical pain, a composition comprising a TRPV 1 antagonist can be topically administered before an activity with an increased risk of external trauma (such as an impact), injury, or wound. As is understood by one skilled in the art, an activity with increased risk of an accident, or external trauma, injury or wound encompasses dangerous vocations, combat, and/or sporting activities.

In some embodiments, a composition comprising a TPPV 1 antagonist as disclosed herein is topically administered to a subject in a method of treating an of the following: ischemia including acute cerebral ischemia, pain including chronic pain, neuropathic pain, nociceptive pain, allodynia, inflammatory pain, inflammatory hyperalgesia, cancer pain, dental surgical pain, pelvic hypersensitivity, pelvic pain, inflammation in mammals, especially humans. In some embodiments, a composition comprising a TPPV1 antagonist is topically administered to a subject for the treatment of pain, particularly for the treatment of any one or more of nociceptive pain or inflammatory pain. In some embodiments, the pain is neuropathic pain. In some embodiments, the pain is associated with phantom limb pain, e.g., pain associated with the removal of a part of the body, e.g., limb, or removal of a body part other than the limbs, e.g. after amputation of the breast, extraction of a tooth (phantom tooth pain) or removal of an eye (phantom eye syndrome).

In some embodiments, compositions, methods and kits comprising a TPPV1 antagonist as disclosed herein are useful as an antidote to capsaicin. In some embodiment, where capsaicin is applied for the treatment of pain with the aim of desensitization of the neurons, and where capsaicin is either effective and/or results in additional pain, topical application of a TPPV1 antagonist can be used to neutralize the capsaicin. In some embodiments, where capsaicin spray (which is more commonly known as pepper spray) is used to control behavior (e.g., in prisons, policing, riot or crowd control, and as a self-defense tool), topical administration of a TPPV1 antagonist as disclosed herein can be used to neutralize, or serve as an antidote, to the capsaicin spray once the person or animal whom was sprayed with the capsaicin spray is under control or otherwise restrained.

Topical administration of TRPV1 antagonists as disclosed herein, including but not limited to those specified in the examples, can be used to treat pain as demonstrated by Nolano, M. et al., Pain 81 (1999) 135; Caterina, M. J. and Julius, D., Annu. Rev. Neurosci. 24, (2001) 487-517; Caterina, M. J. et al., Science 288 (2000) 306-313; Caterina, M. J. et al., Nature 389 (1997) 816-824.

Topical administration of TRPV1 antagonists as disclosed herein, including but not limited to those specified in the examples, can be used to treat inflammatory thermal hyperalgesia as demonstrated by Davis, J. et al., Nature 405 (2000) 183-187.

In some embodiments, topical administration of TRPV1 antagonists as disclosed herein, can be in a method for treating a skin disorder that is associated with, or causes a tissue injury or wound. In some embodiments, a skin disorder that is associated with, or causes a tissue injury or wound is selected from any one or more of: dermatitis, atopic dermatitis, psoriasis, eczema, rosacea, acne, seborrheic dermatitis, rubeola (measles), cold sore, necrotizing fasciitis, cellulitis, impetigo, decubitus ulcer, erysipelas, diaper rash, blisters, bed sores, cancer sore, dyshidrotic eczema, ingrown nail, sebaceous cyst, lichen planus, pilonidal sinus, shingles, chicken pox, skin ulcers, skin infection. In some embodiments, a skin disorder that is associated with, or causes a tissue injury or wound is selected from any one or more of: infected hair follicle (folliculitis), furuncle, carbuncles, impetigo, pressure sores and ulcers (e.g., bed sores), cellulitis, Necrotizing fasciitis, insect bite. In some embodiments, a skin disorder that is associated with, or causes a tissue injury or wound is selected from any one or more of: cellulitis, gangrene, infection with a flesh-destroying or flesh-eating bacteria. In some embodiments, a skin disorder that is associated with, or causes a tissue injury or wound is a chemical injury, a chronic venus ulcer.

### Kits

The disclosure further provides kits that can simplify the handling and topical administration of a TRPV1 antagonist as disclosed to an animal, e.g., a mammal, e.g., a human subject.

Also provided are kits that find use in practicing the subject methods, as described above. For example, kits for practicing the subject methods may include a quantity of the topical formulated TRPV1 antagonist, present in unit dosages, e.g., ampoules, or a multi-dosage format. As such, in certain embodiments, the kits may include one or more unit dosages (e.g., ampoules) of the topical formulated TRPV1 antagonist. The term “unit dosage”, as used herein, refers to physically discrete units suitable as unitary dosages for human and animal subjects, each unit containing a predetermined quantity of the subject emulsion formulation calculated in an amount sufficient to produce the desired effect. The amount of the unit dosage of the subject topical formulated TRPV1 antagonist depends on various factors, such as the particular active TRPV1 antagonist employed, the effect to be achieved, and the pharmacodynamics associated with the active TRPV1 antagonist in the subject. In yet other embodiments, the kits may include a single multi dosage amount of the topical formulated TRPV1 antagonist.

In one embodiment, a kit of the disclosure comprises a unit dosage form of a TRPV1 antagonist as disclosed herein. In one embodiment, the unit dosage form comprises a first container, which can be sterile, containing an effective amount of a TRPV1 antagonist as disclosed herein and a pharmaceutically acceptable carrier or excipient. The kit can further comprise a label or printed instructions instructing the use of a TRPV1 antagonist as disclosed herein to treat a tissue injury or wound. The kit can further comprise a unit dosage form of a second therapeutic agent, for example, a second container containing an effective amount of the second therapeutic agent and a pharmaceutically acceptable carrier or excipient. In another embodiment, the kit comprises a container containing an effective amount of a TRPV1 antagonist as disclosed herein, an effective amount of a second therapeutic agent and a pharmaceutically acceptable vehicle, carrier, or excipient. Examples of second therapeutic agents include, but are not limited to, those listed above.

In various embodiments, a kit is provided that may include additional parts along with the topical applicator device, alone or combined together with the composition comprising at least one TRPV1 antagonist to be used for topical application of the TRPV1 antagonist. The kit may include the topical applicator device for localized/topical delivery of the TRPV1 antagonist in a first compartment, where the topical applicator device comprises in it the composition comprising the TRPV1 antagonist. In alternative embodiments, the topical applicator may not comprise the TRPV1 antagonist, and the first compartment, or a second compartment can comprises a canister or bottle comprising the TRPV1 antagonist, and any other device or instrument needed to add the composition comprising the TRPV1 antagonist to the topical applicator device. A second or third compartment may include gloves, drapes, wound dressings and other procedural supplies for maintaining sterility of the treatment and/or tissue injury, wound or trauma site, as well as an instruction booklet. The kit can comprise more than one topical applicator device, either comprising or not comprising the composition comprising the TRPV1 antagonist (e.g., the composition comprising the TRPV1 antagonist can be separate). Each tool, e.g., topical applicator device may be separately packaged in a plastic pouch that is radiation sterilized. A cover of the kit may include illustrations of the topical administration procedure and a clear plastic cover may be placed over the compartments to maintain sterility.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., one or more pieces of paper on which the information is printed, in the packaging of the kit, in a package insert, etc. The instructions may be present on a computer readable medium, e.g., diskette, CD, DVD, etc., on which the information has been recorded. The instructions may be present on a website, which may be used via the internet to access the information at a removed site. Other convenient means are possible and may be included in the kits.

Such kits are useful for pain management or treating a subject after an accident or injury “in the field” or at a remote location, by a medical team or by any person while waiting for emergency services and medical teams to arrive at the scene of the accident or injury. Accordingly, kits as disclosed herein are suitable for, for example, but not limited to, kits for topically administering a therapeutically effective amount of a TRPV1 antagonist to a subject as disclosed herein during field surgery, or combat casualty care, or combat anesthesia. As such, kits as disclosed herein are useful to be carried by emergency medical personnel, e.g., first responders, EMTs, ambulance staff, firepersons, police, as well as mountain rescue teams, soldiers and combat personnel, and the like. It is also appreciated that the kits are useful for nurses, doctors and surgical personnel, e.g., for preventing pain prior to surgical incision, and for the treatment of post-surgical pain.

Additionally, the kits are useful for personal use, e.g., in first aid kits. In some embodiments, such kits as disclosed herein are useful where there a risk of an accident or external trauma or accident, or where a subject is engaged in a dangerous vocation, combat, and/or sporting activities. Examples of first aid kits can be, for example, but not limited to first aid kits for mountaineering, hiking, for use on boats, mountain biking, skiers, cycling, in cars and trucks, at schools, therefore allowing a TRPV1 antagonist to be topically administered to an injured subject, where topical administration is by the injured subject themselves or with the assistance of different person, in the event of an accident where the subject has a trauma, tissue injury or wound.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of the inhibitor, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

The following examples are included to demonstrate preferred embodiments of the invention. It should be appreciated by those of skill in the art that the techniques disclosed in the examples which follow represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

Having generally described this invention, the same will become more readily understood by reference to the following specific examples which are included herein for purposes of illustration only and are not intended to be limiting unless otherwise specified.

Some embodiments of the invention are listed in the following paragraphs:
1. A composition comprising: (i) a pharmaceutical composition comprising a transient receptor potential vanilloid (TRPV1) antagonist formulated for topical application; and (ii) an applicator for topical administration of the pharmaceutical composition.
2. The composition of paragraph 1, wherein the TRPV1 antagonist is formulated as a powder, ointment or salve, aerosol, gel, emulsion, foam, cream or lotion.
3. The composition of paragraph 1, wherein the applicator is a wipe, a spray, pump or aerosol bottle, a dermal patch, a matrix, a brush, a dressing or sponge, a syringe applicator, optionally calibrated, an ampule, a bottle with irrigating nozzle, pipette dropper or liquid dispenser, a topical spreading applicator, a tube, a topical dosing device.
4. The composition of any of paragraphs 1 to 3, wherein the pharmaceutical composition comprises at least two or more TRPV1 antagonists.
5. The composition of any of paragraphs 1 to 4 wherein the pharmaceutical composition further comprises a transient receptor potential ankyrin (TRPA1) antagonist.
6. The composition of any of paragraphs 1 to 5, wherein the pharmaceutical composition further comprises any one or more of; an antibiotic, an antimicrobial agent, analgesic, clotting agent, anti-inflammatory agent.
7. The composition of any of paragraphs 1 to 6, wherein the pharmaceutical composition does not comprise an aromatic alcohol.
8. The composition of any of paragraphs 1 to 7, wherein the TRPV1 antagonist is formulated for increased permeability into the skin.
9. The composition of any of paragraphs 1 to 8, wherein the TRPV1 antagonist is formulated for controlled or sustained release.
10. The composition of any of paragraphs 1 to 9, wherein the TRPV1 antagonist is at a lower unit dose than the unit dose used for systemic administration, oral or enteral administration or parenteral administration of the same TRPV1 antagonist.
11. The composition of any of paragraphs 1 to 10, wherein the applicator is capable of administering a unit dose of the TRPV1 antagonist which is sufficient to reduce the acute or nociceptive pain sensation in the subject.
12. The composition of any of paragraphs 1 to 11, wherein topical administration is epicutaneous, administration directly to a wound, transdermal, ophthalmic, nasal drops, vaginal, rectal suppositories.
13. The composition of any of paragraphs 1 to 12, wherein the TRPV1 antagonist is stable at room temperature.
14. The composition of paragraph 1, for use in the immediate treatment of acute pain or nociceptive pain of a subject in the field, or at a location where the subject was injured.
15. The composition of paragraph 14, wherein the immediate treatment is within 0 hours and 3 hours of injury to the subject.
16. The composition of any of paragraphs 1 to 15, wherein the pharmaceutical composition comprising the TRPV1 antagonist is present in the applicator, allowing immediate topical administration of the pharmaceutical composition to a subject.
17. The composition of any of paragraphs 1 to 16, present in a kit comprising instructions for use by a non-medically trained person.
18. The composition of any of paragraphs 1 to 15, wherein the pharmaceutical composition further comprises an effective amount of a second therapeutic agent.
19. The composition of paragraph 18, wherein the second therapeutic agent is selected from any of: ketamine, amitriptyline, baclofen, lidocaine, diclofenac, doxepepin and clonidine.
20. The composition of paragraph 18 or 19, wherein the effective amount of the second therapeutic agent is lower than the effective amount of the second therapeutic agent when administered topically by itself or in the absence of the TRPV1 antagonist.
21. A method for treating acute pain or nociceptive pain in a subject, comprising topically administering at, or near a site of tissue injury in the subject, a pharmaceutical composition comprising a TRPV1 antagonist.
22. The method of paragraph 21, wherein the TRPV1 antagonist is formulated for topical application.
23. The method of any of paragraphs 21 to 22, wherein topical administration of the TRPV1 antagonist occurs between 0 hours and 3 hours of the occurrence of tissue injury.
24. The method of any of paragraphs 21 to 23, wherein topical administration is selected from the group consisting of: epicutaneous administration directly to a tissue injury, transdermal administration, ophthalmic administration, nasal drops, or administered via vaginal or rectal suppositories.
25. The method of any one of paragraphs 21 to 24, wherein the tissue injury is an incision, a surgical incision, a wound, a burn, a tissue trauma, an abrasion, a laceration, a puncture or avulsion.
26. The method of any one of paragraphs 21 to 25, wherein the tissue injury is not a burn or not an eye injury.
27. The method of any one of paragraphs 21 to 26, wherein the acute pain or nociceptive pain is post-surgical pain or phantom limb pain.
28. The method of any one of paragraphs 21 to 27, wherein the pharmaceutical composition comprising the TRPV1 antagonist is not washed out or rinsed from the site of the tissue injury.
29. The method of paragraph 25, wherein the pharmaceutical composition comprising a TRPV1 antagonist is topically administered to the skin immediately prior to a surgical incision.
30. The method of any one of paragraphs 21 to 29, wherein the subject is a human.
31. The method of any one of paragraphs 21 to 29, wherein the subject is a domestic animal or a commercial animal.
32. The method of paragraph 21, using the composition of any of paragraphs 1 to 20.
33. A topical delivery device comprising a container comprising a pharmaceutical composition comprising a TRPV1 antagonist, and an applicator for topical administration of the TPRV1 antagonist to the site of a wound or tissue injury in a subject.
34. The topical delivery device of paragraph 33, wherein the container is a bottle and the applicator is a brush, spray, pump, pipette nozzle, pipette dropper dispenser.
35. The topical delivery device of paragraph 33, wherein the container allows for pressed-air assisted spraying or aerosol spraying of the pharmaceutical composition comprising a TRPV1 antagonist.
36. The topical delivery device of any of paragraphs 33-35, wherein the composition comprises any of paragraphs 1-20.
37. A kit comprising a pharmaceutical composition comprising a transient receptor potential vanilloid (TRPV1) antagonist formulated for topical application; and an applicator for topical administration of the pharmaceutical composition.
38. A kit comprising the topical delivery device of paragraphs 33-36, and instruction for use.

### EXAMPLES

As TRPV1 antagonists are administered systemically, clinical use of TRPV1 antagonists has been halted or significantly hampered, as discussed in Gavva “Setbacks in the Clinical Development of TRPV1 antagonists: What Next?” The Open Drug Discovery Journal, 2009; 1 (1-35). Here, the inventors have discovered that topical application of TRPV1 antagonists can be used for reducing pain caused by wounds and incisions.

Accordingly, the examples presented herein generally relate to compositions, kits and methods for topical application of a TRPV1 antagonist for the treatment of a trauma, tissue injury or wound. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

All human and animal experiments were performed according to protocols following institutional IACUC and IRB guidelines. All animal experiments were performed with male mice at least 8 weeks of age.

### Example 1

Several species and mammals, including humans lick their wounds¹⁻³. The widespread instinctive drive for this acute behavior suggests it is an evolutionarily conserved adaptation. The inventors assessed if this licking of the wounds provides comfort to the injured mammal by reducing pain through peptides in saliva that inhibit the transduction of noxious stimuli by high threshold nociceptor sensory neurons. Here, the inventors show that saliva from several human donors was able to inhibit the TRPV1 ion channel that mediates heat and capsaicin-evoked pain. Further analysis of saliva fractions identified peptides belonging to the opiorphin family⁴⁻⁷ as endogenous inhibitors for the channel Opiorphin was selective for TRPV1 at physiologically relevant concentrations of 1 μM and inhibited it sufficiently in a non-competitive way via its external pore turret domain to reduce the acute pain-like response capsaicin evokes in mice. Topical application of opiorphin, like the selective TRPV1 antagonist AMG9810, eliminated the nocifensive licking behavior in mice produced by skin incision, highlighting the importance of pain in initiating wound licking. Indeed, wound licking behavior was completely abolished in mice lacking either TRPV1 receptors or TRPV1+ neurons further highlighting the importance of TRPV1 in wound pain. We conclude that endogenous TRPV1 antagonists in saliva, and the analgesia they enable, explain why we lick wounds.

Many animal species lick their wounds. Indeed, licking wounds, boils and burns by humans was quite common until the early 20^{th} century and is still prevalent in isolated communities¹⁻³. Why are wounds licked? Explanations have included the blood clotting, anti-inflammatory and antibiotic effects of saliva⁸⁻¹¹. However, an animal is unlikely to be motivated to lick wounds for a potential wound healing benefit that manifests only in the long-term, unless this was either inbuilt or acutely rewarding, and the latter we surmised could include immediate relief of pain. To test the possibility that saliva may inhibit the TRPV1 heat pain receptor¹²⁻¹⁴ in nociceptors, we collected samples of whole saliva from four human donors and ran a screen on primary murine dorsal root ganglion (DRG) neurons.

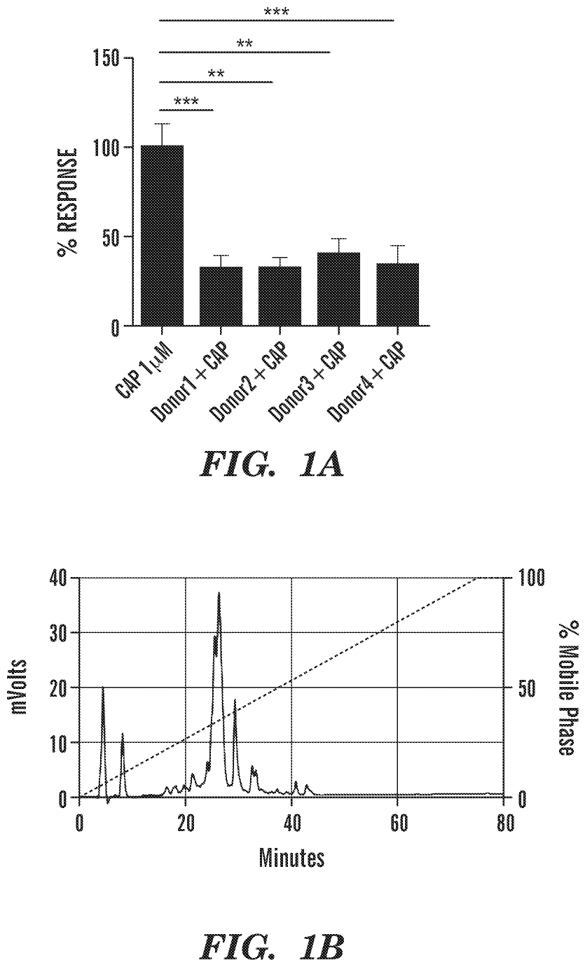

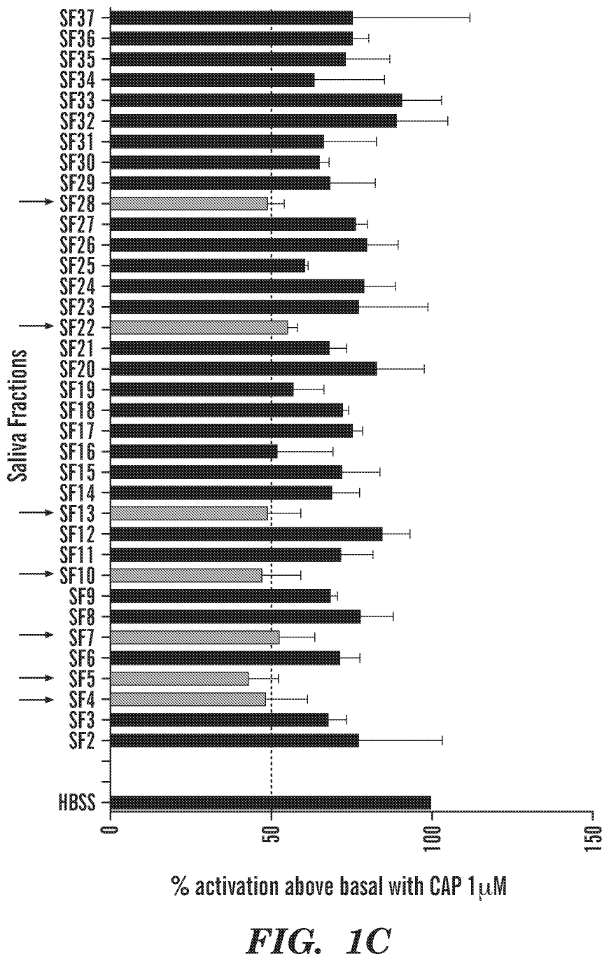

All four samples significantly inhibited the calcium flux induced by 1 μM capsaicin by >70±5% (FIG. 1A; p<0.005, One-way ANOVA and Bonferroni posthoc test) indicating the likely presence in saliva of endogenous TRPV1 antagonists. To identify if the antagonists were peptides, we purified saliva through a 30 kDa filter to eliminate large proteins and then used reverse-phase HPLC to fractionate the samples (FIG. 1B). The first fraction mainly contained salts and was eliminated from further analysis. The resultant 37 1 mL-fractions were dried, reconstituted in HBSS and screened on HEK cells transiently transfected with TRPV1 (FIG. 1C).

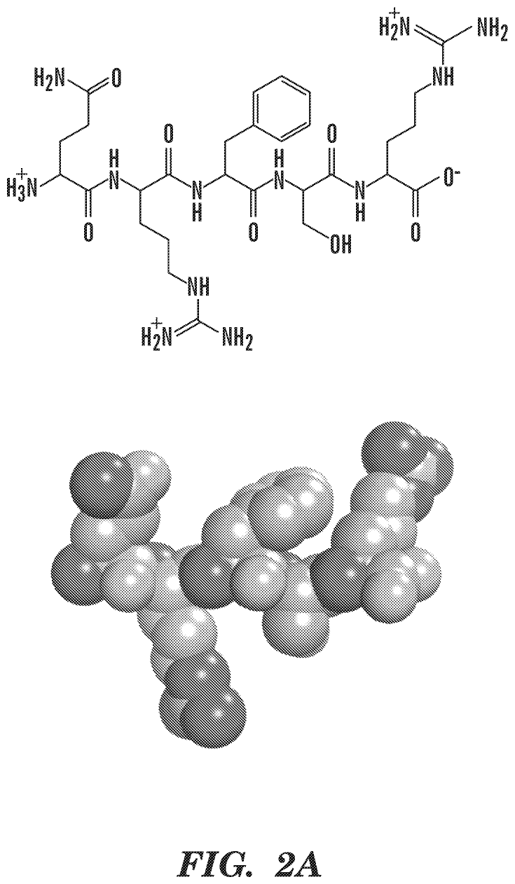

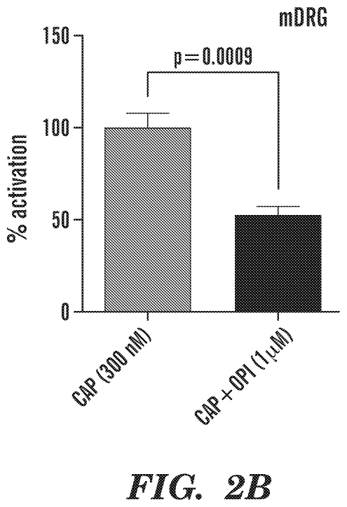

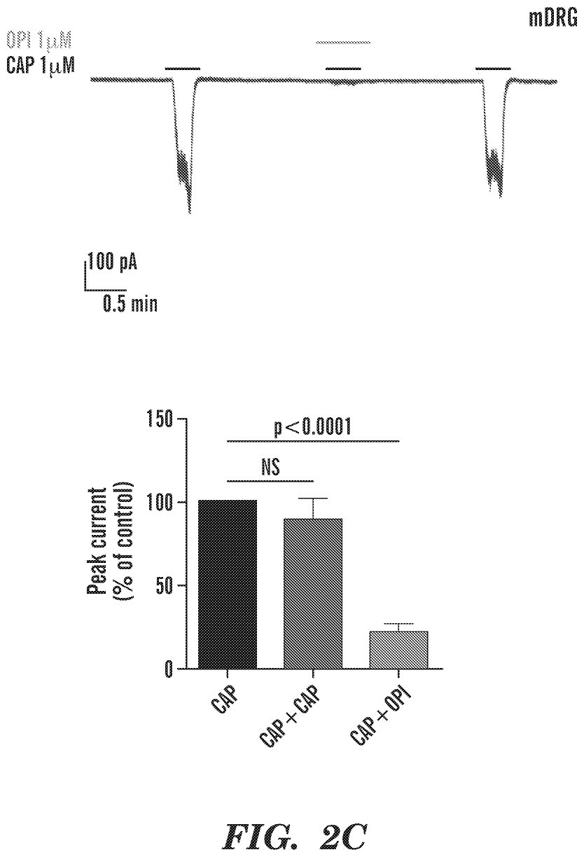

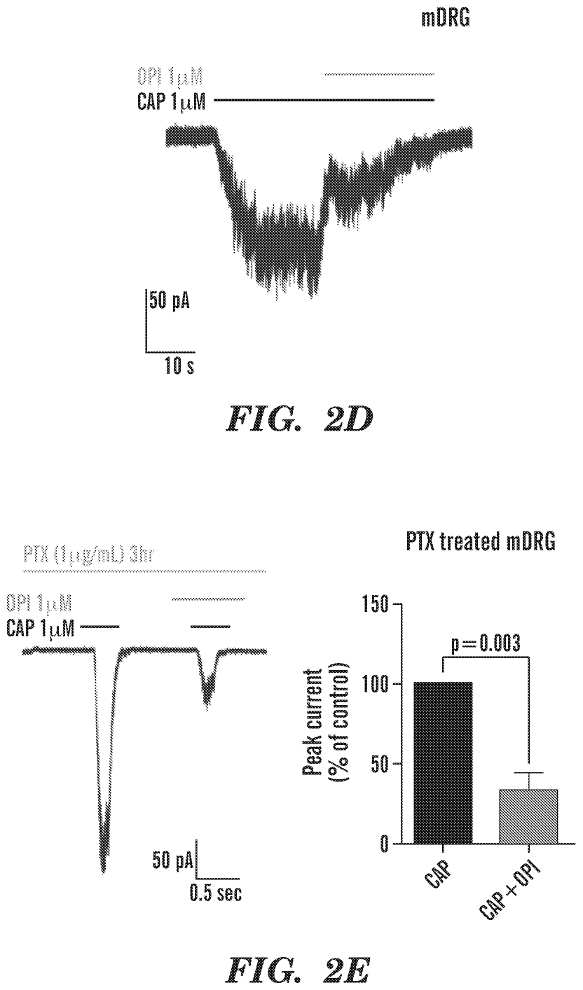

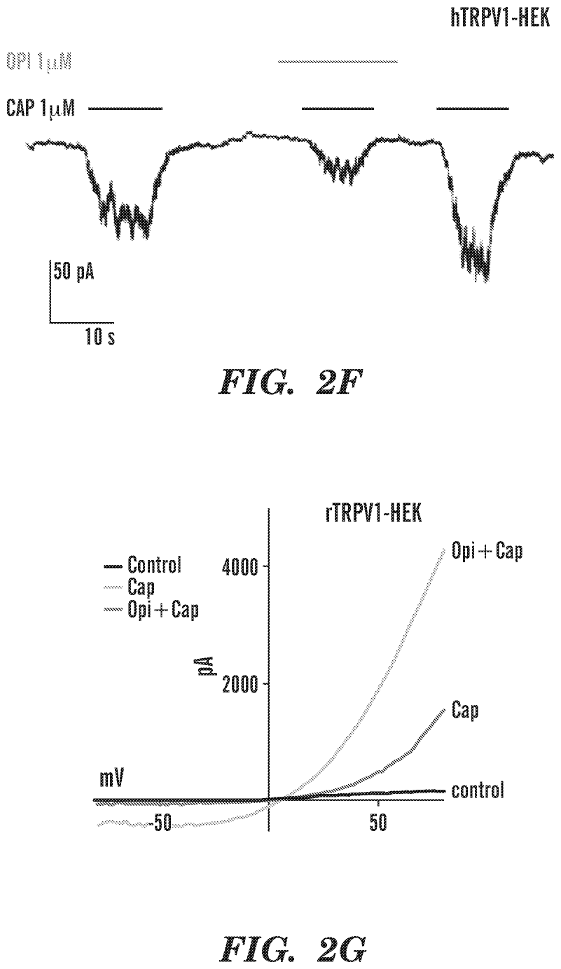

Seven fractions produced >50% inhibition of capsaicin-induced calcium fluxes (FIG. 1C). MS/MS analysis of three of the seven active fractions revealed >30,000 unique peptides. Given this enormous number, we decided to focus on sets of candidate peptides with a reported analgesic annotation. One of these was opiorphin (QRFSR (SEQ ID NO: 3), present in fraction 10), a pentapeptide from the N-terminal region of PROL1 (FIG. 2A), has antinociceptive properties produced by inhibiting ectopeptidases in the spinal dorsal horn⁴⁻⁷.

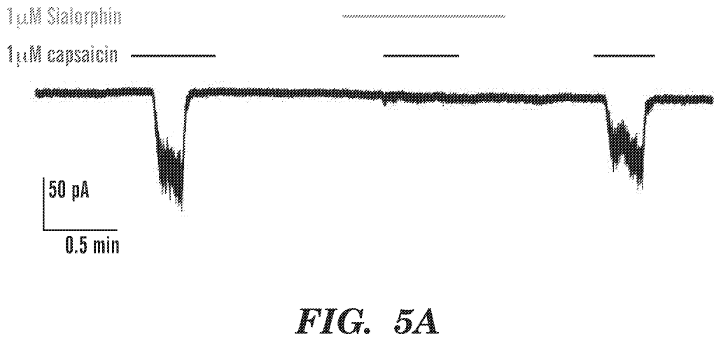

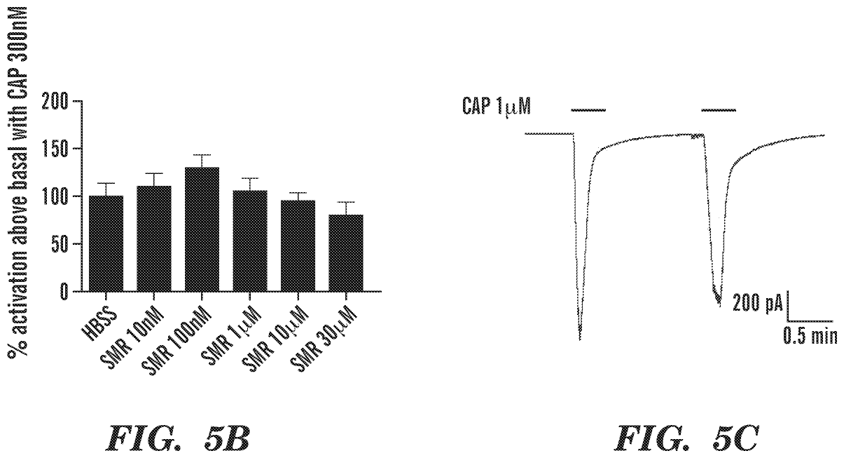

Here, the inventors first tested the efficacy of human opiorphin (FIG. 2A) on capsaicin-evoked calcium flux in primary mouse DRG neurons. Opiorphin (1 μM) strongly inhibited the capsaicin-induced calcium flux in a FLIPR assay (˜50% inhibition, p<0.05, t-test) (FIG. 2B). The inventors then tested opiorphin's ability to block capsaicin-induced currents in primary murine DRG neurons using whole cell patch clamp. Opiorphin (1 μM) effectively eliminated these calcium currents (FIG. 2C) in a reversible fashion. Furthermore, opiorphin (1 μM) inhibited capsaicin currents when applied 10 s into the application of capsaicin suggesting that it can inhibit the receptor even while the channel is in its active conformation (FIG. 2D). Since opiorphin belongs to a family of three peptides that have been implicated in antinociception and erectile dysfunction⁴⁻⁷, we tested each of them for potential activity at TRPV1. The rat peptide sialorphin (QHNPR, SEQ ID NO: 4) also blocked capsaicin-induced currents but the SMR3 peptide (QRGPR; SEQ ID NO: 5) had no such activity in DRG neurons (FIGS. 5A and 5B).

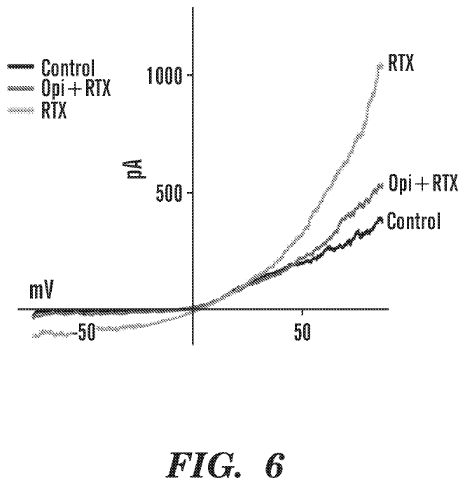

To eliminate the possibility of any G protein coupled receptors (GPCRs) such as opioid receptors playing a role in the inhibitory effect of opiorphin in DRG neurons, the inventors incubated the neurons for 3 hours with pertussis toxin (PTX, 1 μg/mL), which inhibits Gᵢ protein function, and then applied capsaicin and opiorphin. Both the agonist effect of capsaicin and ability of opiorphin to block capsaicin-evoked currents remained intact (FIG. 2E), showing that no Gᵢ coupled GPCRs are involved. Indeed, opiorphin inhibited capsaicin-induced currents in HEK293 cells transiently expressing hTRPV1 (FIG. 2F) suggesting that inhibition occurs directly at the TRPV1 receptor, and opiorphin inhibited both mouse and human heterologously expressed channels. Opiorphin (1 μM) also antagonized capsaicin-evoked currents at all voltages tested (FIG. 2G) in HEK293 cells transiently expressing rat TRPV1, and inhibited the action of another TRPV1 agonist, the vanilloid toxin resifineratoxin (RTX, 100 nM) (FIG. 6).

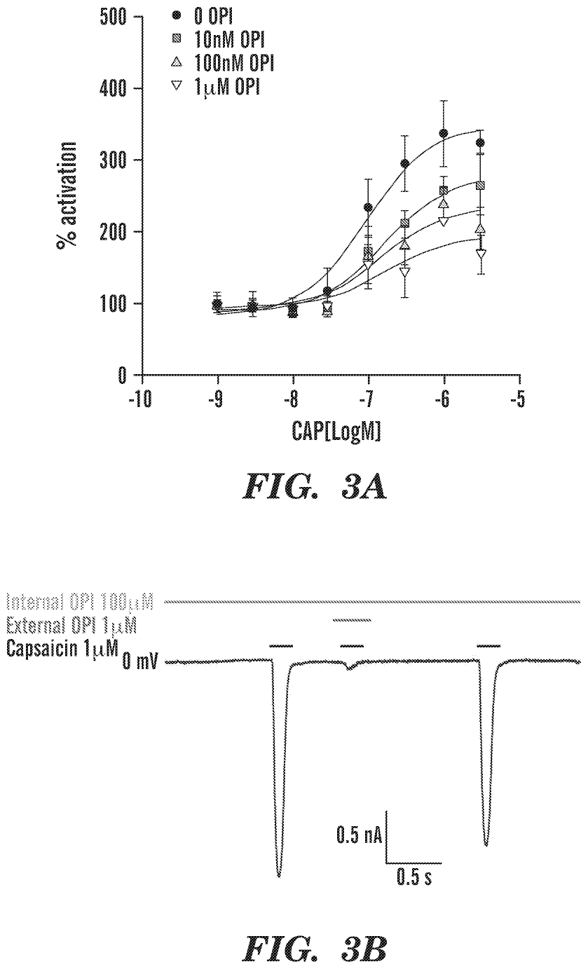

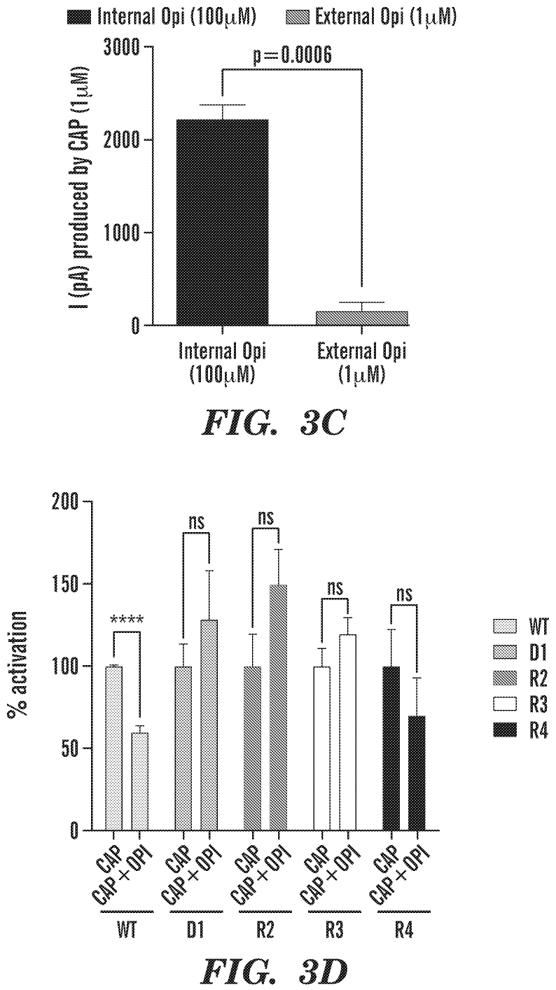

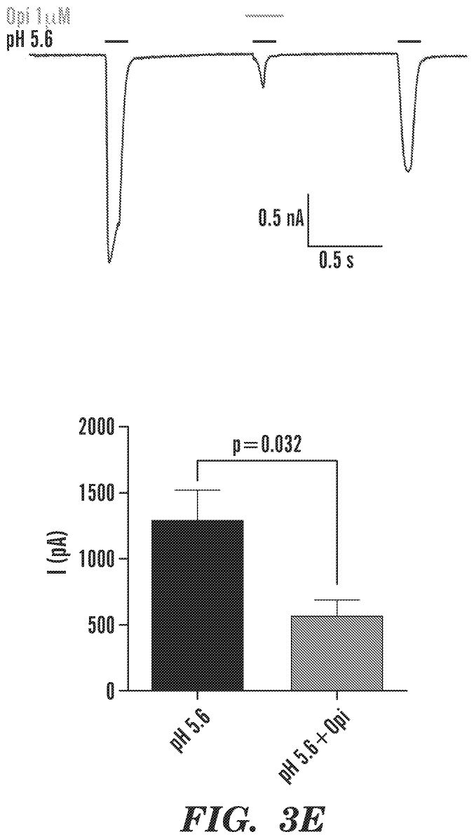

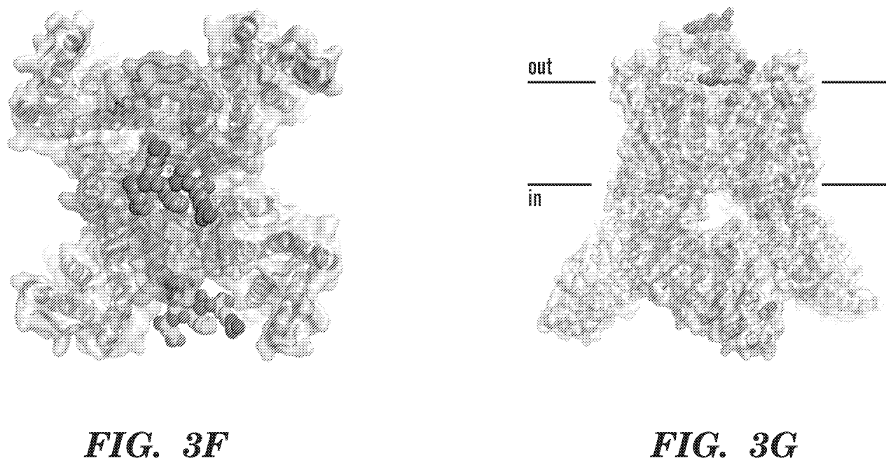

Opiorphin is expressed in the range of 115 nM-1.57 μM in human saliva¹⁵. The inventors used 1 μM as a standard concentration for all the in vitro experiments but in concentration response experiments on HEK cells transiently expressing rTRPV1, we saw activity at 10 nM of opiorphin (FIG. 3A). Opiorphin reduced the efficacy of capsaicin, but did not produce any rightward-shift of the curve, indicating that opiorphin was inhibiting capsaicin non-competitively (allosterically) via a different site (FIG. 3A). The peak efficacy of capsaicin was reduced to ˜50% by 100 nM opiorphin. This is much lower than the IC₅₀ of opiorphin for Met-Enkephalin breakdown by the ectopeptidase, h-NEP (˜33 μM)⁷. To elucidate the mode and site of opiorphin action on TRPV1 we performed whole cell DRG patch clamp studies where the internal patch solution included 100 μM opiorphin. When capsaicin was applied to these cells, no change in TRPV1 currents was detected (FIGS. 3B and C), however, but when capsaicin was co-applied with 1 μM opiorphin externally, the currents were substantially reduced (FIGS. 3B and C), demonstrating that opiorphin inhibits TRPV1 at an external site.

The inventors next assessed possible external sites on TRPV1 where opiorphin might act, focusing on the external pore region between S5 and S6 helices of TRPV1 (S5-P-S6) where protons, heat and a heterodimeric tarantula toxin (DkTx) activate TRPV1¹⁶⁻¹⁹ TRPV1 constructs with deletion and substitution mutations in the pore turret domain (G603-N626 of TRPV1) retain capsaicin activity¹⁷, but these led to loss of opiorphin inhibitory activity (FIG. 3D). It is possible that reversible binding to this region by opiorphin stabilizes the closed conformation of the channel outer pore blocking ion influx via the channel To test this we evaluated the ability of opiorphin to inhibit proton activation of the ion channel (pH 5.6). Opiorphin strongly inhibited proton-induced calcium currents (FIG. 3E), suggesting that it may be a selective inhibitor of the external pore region of TRPV1.

The recently elucidated cryo-EM structure of TRPV1¹⁹⁻²⁰, used a protein construct with deletion of the turret region (residues 604-626). This region is predicted by most secondary structure prediction algorithms to be a disordered loop. Here, the inventors generated a model in which the turret region of each TRPV1 subunit takes on a different loop structure (FIGS. 3F and G). The size of the region suggests that the turrets may form a loose cap over the channel's pore. Alternatively, the loop could extend outward from the center and interact with the S1-S4 bundles or the extracellular leaflet of the bilayer (FIGS. 3F and G). To act as an antagonist, the hydrophilic opiorphin peptide could interact with the turrets to block the pore (FIG. 3F), or could interact with the channel's pore region near the membrane surface (FIG. 3G), analogously to the double-knot toxin (DkTx) agonist^{18,19}.

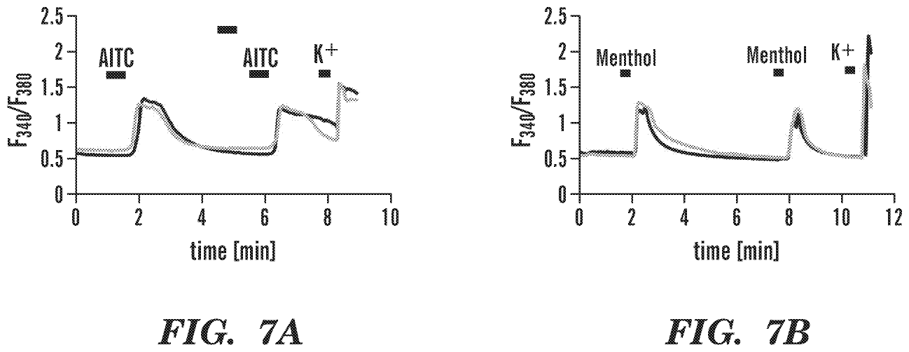

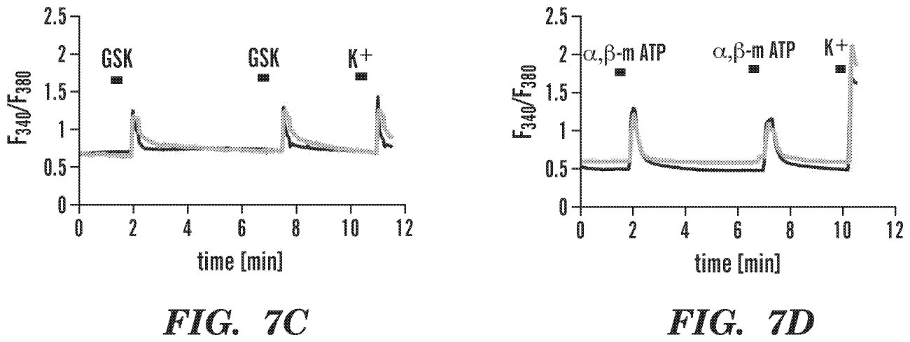

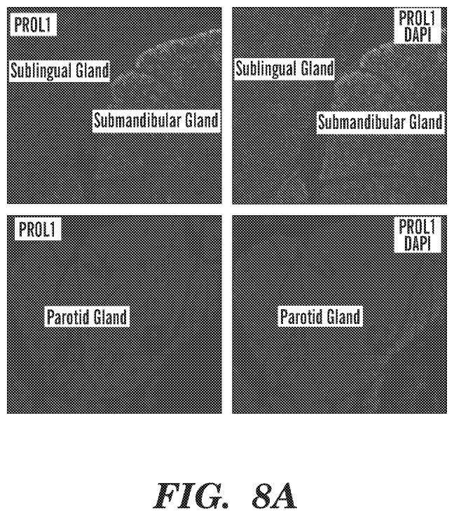

An important hallmark of an effective inhibitor is its selectivity. To this end, the inventors tested if opiorphin antagonized other nociceptor transduction ion channels by co-administering it with their agonists on primary murine DRG neurons. Opiorphin even at a high concentration of 30 μM did not inhibit the calcium flux produced by agonists for TRPA1, TRPM8, TRPV4 or P2X3 channels (FIGS. 7A-7D). To determine which salivary glands express opiorphin we looked at the distribution of its precursor protein, PROL1, in the mouse by immunohistochemistry. The protein was detected in the submandibular gland in acinar cells but not in the parotid or sublingual glands (FIGS. 8A-8B).

### Example 2

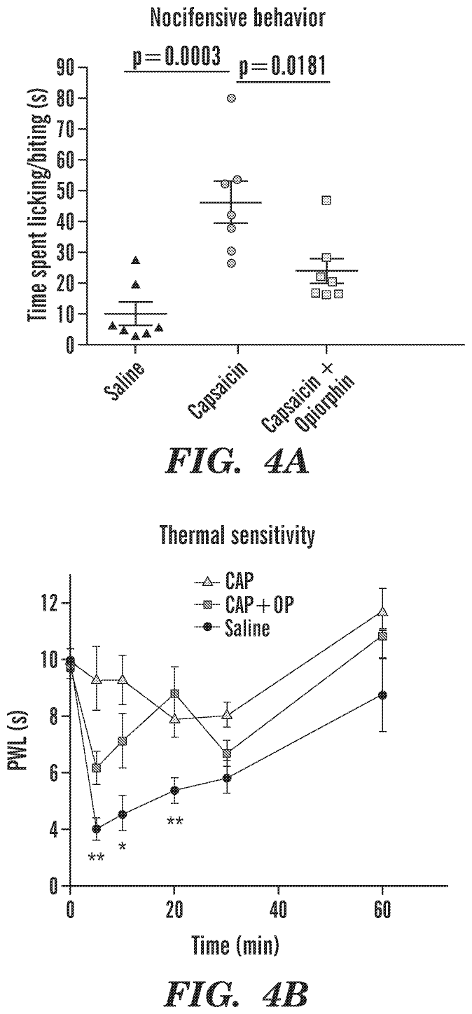

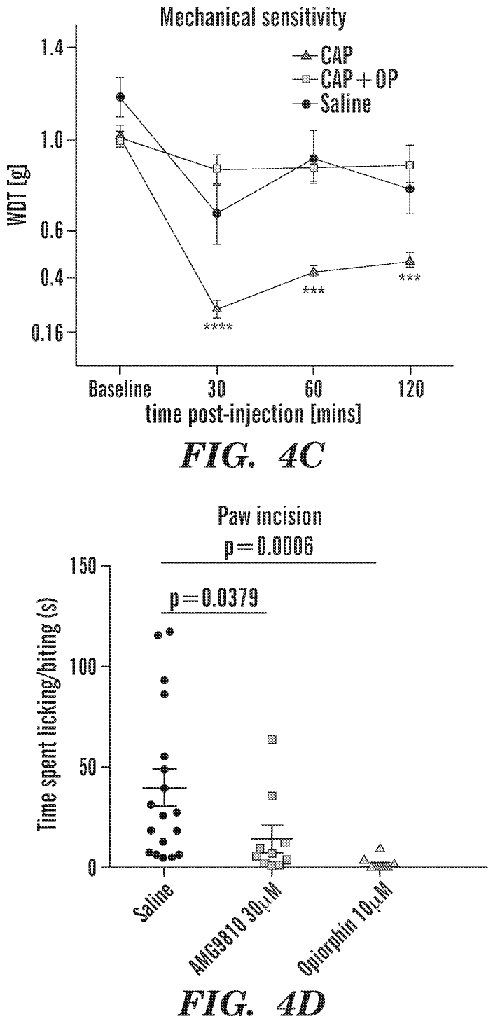

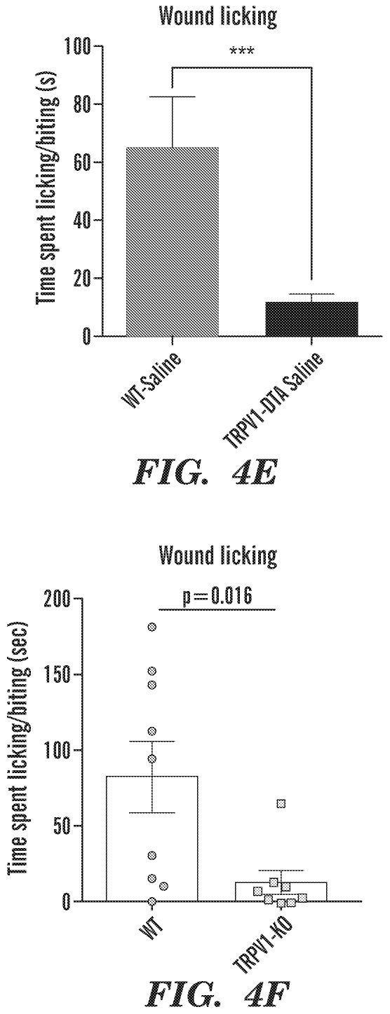

The inventors assessed if opiorphin had analgesic effects on capsaicin-induced nocifensive behavior in vivo. Injection of capsaicin into a paw produces a short bout of biting and licking. It is clear from FIG. 4A that this salivary peptide, opiorphin, significantly reduced the capsaicin-induced licking response (FIG. 4A) (both ligands used at 5 nmol/mouse). Surprisingly, Opiorphin also reduced the thermal and mechanical hypersensitivity that follows such a capsaicin injection (FIGS. 4B and 4C).

Topical application of the TRPV1 selective antagonist, AMG 9810 (30 μM) reduced the acute licking of a superficial paw incision wound in mice (FIG. 4D), demonstrating that antagonism of this receptor is sufficient to stop the paw licking induced by such an injury, an effect which is also produced by topical application of opiorphin (10 μM) (FIG. 4D). For technical reasons, the inventors selected a single application of a high concentration (10 μM opiorphin) for 5 s versus repeated applications of ˜1 μM over a period of ˜40 s to mimic the exposure that would occur by a mouse naturally licking the wound in this behavioral test (FIG. 4D). The inventors demonstrate that the analgesic properties of saliva resulting from opiorphin are sufficient to cause cessation of wound licking behavior. To further extend this line of enquiry, the inventors tested the wound licking behavior in TRPV1KO and TRPV1-DTA (strain lacking TRPV1+ neurons) mice using the incision model on the left hindpaw (FIGS. 4E and F). Both strains showed significantly attenuated wound licking behaviors when compared with their littermate WT controls suggesting that TRPV1 plays a central role in wound pain transmission.

In conclusion, we have discovered a salivary peptide that is an endogenous inhibitor of TRPV1 and that pain-relief consequent on blocking TRPV1 is the driving force for wound licking behavior. Longer lasting benefits of such licking, such as removal of debris, antimicrobial actions and wound healing may be facilitated by this endogenous analgesia, which would also contribute to widespread addition of capsaicin to foods where its pain-producing action in the mouth must be attenuated by saliva.

### Example 3

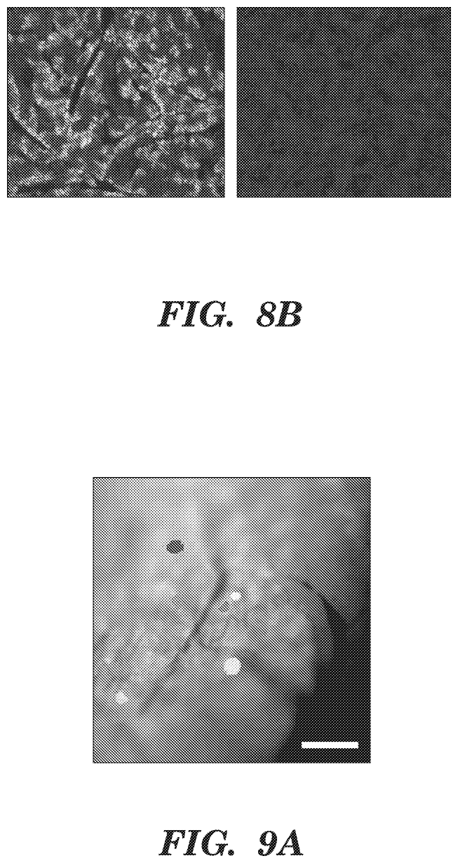

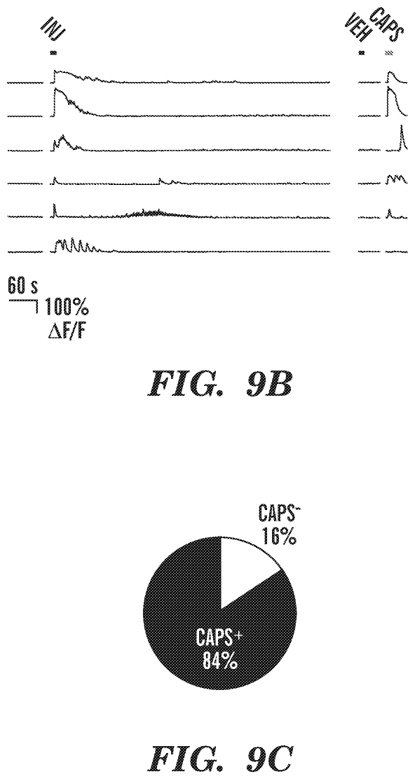

The inventors assessed effect of topical TRPV1 agonist capsaicin applied after acute injury and wound in a mouse model in vivo. FIGS. 9A-9C shows that after acute injury/wound, most of the neurons activated in vivo are TRPV1-positive. FIGS. 9A-9C shows data of murine neurons imaged in the intact, live animals where a surgical injury was performed to identify neuronal activation. Capsaicin, a TRPV1 agonist, was later applied to the same region to determine that an unexpected high number of neurons, ˜86% of neurons activated by injury were also TRPV1-positive. Accordingly, the inventors have demonstrated that because the majority of injured neurons are TRPV1-positive, topical application of TRPV1 antagonists work well for pain relief when applied topically to wounds in mammals.

### REFERENCES

The references cited in the background and throughout the specification are incorporated herein in their entirety by reference.
1. Bergen, F., Animal and plant lore. (Houghton, Mifflin and company; [etc., Boston and New York, 1899).
2. Seoighe, D. M., Baker, J. F., & Conroy, F., Licking as an out-of-hospital burns treatment-An isolated cultural phenomenon? Burns 37 (2), 348-350.
3. Verrier, L., Dog licks man. Lancet 1 (7647), 615 (1970).
4. Rougeot, C. et al., Sialorphin, a natural inhibitor of rat membrane-bound neutral endopeptidase that displays analgesic activity. Proc Natl Acad Sci USA 100 (14), 8549-8554 (2003).
5. Tong, Y., Tar, M., Melman, A., & Davies, K., The opiorphin gene (ProLl) and its homologues function in erectile physiology. BJU Int 102 (6), 736-740 (2008).
6. Javelot, H., Messaoudi, M., Gamier, S., & Rougeot, C., Human opiorphin is naturally occurring antidepressant acting selectively on enkephalin-dependent delta-opioid pathways. J Physiol Pharmacol 61 (3), 355-362.
7. Wisner, A. et al., Human Opiorphin, a natural antinociceptive modulator of opiod dependent pathways. Proc Natl Acad Sci USA 103 (47), 17979-17984 (2006).
8. Bodner, L., Effect of parotid submandibular and sublingual saliva on wound healing in rats. Comp Biochem Physiol A Comp Physiol 100 (4), 887-890 (1991).
9. Dodds, M. W., Johnson, D. A., & Yeh, C. K., Health benefits of saliva: a review. J Dent 33 (3), 223-233 (2005).
10. Brand, H. S., Ligtenberg, A. J., & Veerman, E. C., Saliva and wound healing. Monogr Oral Sci 24, 52-60.
11. Noguchi, S., Ohba, Y., & Oka, T., Effect of salivary epidermal growth factor on wound healing of tongue in mice. Am J Physiol 260 (4 Pt 1), E620-625 (1991).
12. Caterina, M. J. et al., The capsaicin receptor: a heat-activated ion channel in the pain pathway. Nature 389 (6653), 816-824 (1997).
13. Caterina, M. J. et al., Impaired nociception and pain sensation in mice lacking the capsaicin receptor. Science 288 (5464), 306-313 (2000).
14. Mishra, S. K., Tisel, S. M., Orestes, P., Bhangoo, S. K., & Hoon, M. A., TRPV1-lineage neurons are required for thermal sensation. EMBO J30 (3), 582-593.
15. Dufour, E., Villard-Saussine, S., & Mellon., L., R., Jouannet, P., Unheheuer M N & Rougeot C, Opiorphin Secretion Pattern in Healthy Volunteers: Gender Difference and Organ Specificity. Biochemistry & Analytical Biochemistry 2 (3), 136-147 (2013).
16. Jordt, S. E., Tominaga, M., & Julius, D., Acid potentiation of the capsaicin receptor determined by a key extracellular site. Proc Natl Acad Sci USA 97 (14), 8134-8139 (2000).
17. Cui, Y. et al., Selective disruption of high sensitivity heat activation but not capsaicin activation of TRPV1 channels by pore turret mutations. J Gen Physiol 139 (4), 273-283.
18. Bohlen, C. J. et al., A bivalent tarantula toxin activates the capsaicin receptor, TRPV1, by targeting the outer pore domain. Cell 141 (5), 834-845.
19. Cao, E., Liao, M., Cheng, Y., & Julius, D., TRPV1 structures in distinct conformations reveal activation mechanisms. Nature 504 (7478), 113-118.
20. Liao, M., Cao, E., Julius, D., & Cheng, Y., Structure of the TRPV1 ion channel determined by electron cryo-microscopy. Nature 504 (7478), 107-112.
21. Gavva “Setbacks in the Clinical Development of TRPV1 antagonists: What Next?” The Open Drug Discovery Journal, 2009; 1 (1-35)
22. Gibson et al., A Randomised Trial Evaluating the Effects of the TRPV1 Antagonist SB705498 on Pruritus Induced by Histamine, and Cowhage Challenge in Healthy Volunteers. PLOS one; 2014, 9(7); e100610;
23. Osamu Yamanaka, Ai Kitano-Izutani, Katsuo Tomoyose and Peter S. Reinach. Pathobiology of wound healing after glaucoma filtration surgery. BMC Ophthalmology, 201515 (Suppl 1):157
24. Hesselink JMK (2016) Topical Analgesics: Critical Issues Related to Formulation and Concentration. J Pain Relief 5:274. doi: 10.4172/2167-0846.1000274
25. Hesselink J M K, et al., Bottlenecks in the development of topical analgesics: molecule, formulation, dose-finding, and phase III design, J Pain Res. 2017; 10: 635-641.
26. Kopsky et al., Analgesic effects of topical ketamine. Minerva Anestesiol. 2015 April; 81(4):440-9. Epub 2014 May 22.
27. Mercadante S, Topical amitriptyline and ketamine for the treatment of neuropathic pain. Expert Rev Neurother. 2015;15(11):1249-53. doi: 10.1586/14737175.2015.1101347. Epub 2015 Oct. 21.
28. Sawynok J. Topical analgesics for neuropathic pain: preclinical exploration, clinical validation, future development. Eur J Pain. 2014 Apr;18(4):465-81. doi: 10.1002/j.1532-2149.2013.00400.x. Epub 2013 Sep. 24.
29. Sawynok J. Topical analgesics for neuropathic pain in the elderly: current and future prospects. Drugs Aging. 2014 Dec;31(12):853-62. doi: 10.1007/s40266-014-0218-9.
30. Casale R, Symeonidou Z, Bartolo M. Topical Treatments for Localized Neuropathic Pain. Curr Pain Headache Rep. 2017 Mar; 21(3):15. doi: 10.1007/s11916-017-0615-y.
31. Allegri M, Baron R, Hans G, Correa-Illanes G, Mayoral Rojals V, Mick G, Serpell M. A pharmacological treatment algorithm for localized neuropathic pain. Curr Med Res Opin. 2016;32(2):377-84. doi: 10.1185/03007995.2015.1129321.
32. Casale R, et al., Topical Treatments for Localized Neuropathic Pain. Curr Pain Headache Rep. 2017 March; 21(3):15. doi: 10.1007/s11916-017-0615-y.
33. Casale R, Mattia C. Building a diagnostic algorithm on localized neuropathic pain (LNP) and targeted topical treatment: focus on 5% lidocaine-medicated plaster. Ther Clin Risk Manag. 2014; 10: 259-68.
34. Zur E. Topical treatment of neuropathic pain using compounded medications. Clin J Pain. 2014;30(1):73-91.
35. Jorge L L, Feres C C, Teles V E. Topical preparations for pain relief: efficacy and patient adherence. J Pain Res. 2010; 4:11-24.
36. Ahmed S U, Zhang Y, Chen L, Cohen A, Hillary K S, Vo T, et al. Effect of 1.5% topical diclofenac on clinical. Neuropathic pain. Anaesthesiology. 2015; 123:191-8.
37. Liebregts R, Kopsky J M, Hesselink J M. Topical amitriptyline in post-traumatic neuropathic pain. J Pain Symptom Manag. 2011; 41:e6-7.
38. Kern K U, Weiser T. Topical ambroxol for the treatment of neuropathic pain. An initial clinical observation. Schmerz. 2015; 29 Suppl 3:S89-96.

1. A composition comprising:
a pharmaceutical composition comprising a transient receptor potential vanilloid (TRPV1) antagonist formulated for topical application; and
an applicator for topical administration of the pharmaceutical composition.

2. The composition of claim 1, wherein the TRPV1 antagonist is formulated as a powder, ointment or salve, aerosol, gel, emulsion, foam, cream or lotion.

3. The composition of claim 1, wherein the applicator is a wipe, a spray, pump or aerosol bottle, a dermal patch, a matrix, a brush, a dressing or sponge, a syringe applicator, optionally calibrated, an ampule, a bottle with irrigating nozzle, pipette dropper or liquid dispenser, a topical spreading applicator, a tube, a topical dosing device.

4. The composition of claim 1, wherein the pharmaceutical composition comprises at least two or more TRPV1 antagonists or an effective amount of a second therapeutic agent, or both.

5. The composition of claim 1, wherein the pharmaceutical composition further comprises a transient receptor potential ankyrin (TRPA1) antagonist.

6. The composition of claim 1, wherein the pharmaceutical composition further comprises any one or more of; an antibiotic, an antimicrobial agent, analgesic, clotting agent, anti-inflammatory agent.

7. The composition of claim 1, wherein the pharmaceutical composition does not comprise an aromatic alcohol.

8. The composition of claim 1, wherein the TRPV1 antagonist is any one or more of:
a. formulated for increased permeability into the skin,
b. formulated for controlled or sustained release, or
c. is at a lower unit dose than the unit dose used for systemic administration, oral or enteral administration or parenteral administration of the same TRPV 1 antagonist.

9.-11. (canceled)

12. The composition of claim 1, wherein topical administration is epicutaneous, administration directly to a wound, transdermal, ophthalmic, nasal drops, vaginal, rectal suppositories.

13.-15. (canceled)

16. The composition of claim 1, wherein the pharmaceutical composition comprising the TRPV 1 antagonist is present in or on the applicator, allowing immediate topical administration of the pharmaceutical composition to a subject.

17. (canceled)

18. (canceled)

19. The composition of claim 4, wherein the second therapeutic agent is selected from any of: ketamine, amitriptyline, baclofen, lidocaine, diclofenac, doxepepin and clonidine.

20. The composition of claim 4, wherein the effective amount of the second therapeutic agent is lower than the effective amount of the second therapeutic agent when administered topically by itself or in the absence of the TRPV 1 antagonist.

21. A method for treating acute pain or nociceptive pain in a subject, comprising topically administering at, or near a site of tissue injury in the subject, a pharmaceutical composition comprising a TRPV 1 antagonist.

22. (canceled)

23. The method of claim 21, wherein topical administration of the TRPV1 antagonist occurs between 0 hours and 3 hours of the occurrence of tissue injury or wherein topical administration is to the skin of the subject immediately prior to a surgical incision.

24. The method of claim 21any of claims 21, wherein the TRPV antagonist is formulated for topical administration is selected from the group consisting of: epicutaneous administration directly to a tissue injury, transdermal administration, ophthalmic administration, nasal drops, or administered via vaginal or rectal suppositories.

25. The method of claim 21, wherein
a. the tissue injury is selected from an incision, a surgical incision, a wound, a burn, a tissue trauma, an abrasion, a laceration, a puncture or avulsion, or
b. the tissue injury is not a burn or not an eye injury, or
c. the acute pain or nociceptive pain is post-surgical pain or phantom limb pain.

26. (canceled)

27. (canceled)

28. The method of claim 21, wherein the pharmaceutical composition comprising the TRPV1 antagonist is not washed out or rinsed from the site of the tissue injury.

29.-31. (canceled)

32. The method of claim 21, using the composition of claim 1.

33. A topical delivery device comprising a container comprising a pharmaceutical composition comprising a TRPV1 antagonist, and an applicator for topical administration of the TPRV1 antagonist to the site of a wound or tissue injury in a subject.

34. The topical delivery device of claim 33, wherein the container
a. is a bottle and the applicator is selected from any of: a brush, spray, pump, pipette nozzle, pipette dropper dispenser, and/or
b. allows for pressed-air assisted spraying or aerosol spraying of the pharmaceutical composition comprising a TRPV1 antagonist.

35.-38 (canceled)


SEQUENCE LISTING (5 sequences)

SEQ ID NO: 1 - protein, 839 aa, Homo sapiens [sequence omitted]

SEQ ID NO: 2 - protein, 1119 aa, Homo sapiens
MKRSLRKMWRPGEKKEPQGVVYEDVPDDTEDFKESLKVVFEGSAYGLQNFNKQKKLKRCDDMDTFFLHYAAAEGQIELMEKITRDSSLEVLHEMDDYGNTPLHCAVEKNQIESVKFLLSRGANPNLRNFNMMAPLHIAVQGMNNEVMKVLLEHRTIDVNLEGENGNTAVIIACTTNNSEALQILLKKGAKPCKSNKWGCFPIHQAAFSGSKECMEIILRFGEEHGYSRQLHINFMNNGKATPLHLAVQNGDLEMIKMCLDNGAQIDPVEKGRCTAIHFAATQGATEIVKLMISSYSGSVDIVNTTDGCHETMLHRASLFDHHELADYLISVGADINKIDSEGRSPLILATASASWNIVNLLLSKGAQVDIKDNFGRNFLHLTVQQPYGLKNLRPEFMQMQQIKELVMDEDNDGCTPLHYACRQGGPGSVNNLLGFNVSIHSKSKDKKSPLHFAASYGRINTCQRLLQDISDTRLLNEGDLHGMTPLHLAAKNGHDKVVQLLLKKGALFLSDHNGWTALHHASMGGYTQTMKVILDTNLKCTDRLDEDGNTALHFAAREGHAKAVALLLSHNADIVLNKQQASFLHLALHNKRKEVVLTIIRSKRWDECLKIFSHNSPGNKCPITEMIEYLPECMKVLLDFCMLHSTEDKSCRDYYIEYNFKYLQCPLEFTKKTPTQDVIYEPLTALNAMVQNNRIELLNHPVCKEYLLMKWLAYGFRAHMMNLGSYCLGLIPMTILVVNIKPGMAFNSTGIINETSDHSEILDTTNSYLIKTCMILVFLSSIFGYCKEAGQIFQQKRNYFMDISNVLEWIIYTTGIIFVLPLFVEIPAHLQWQCGAIAVYFYWMNFLLYLQRFENCGIFIVMLEVILKTLLRSTVVFIFLLLAFGLSFYILLNLQDPFSSPLLSIIQTFSMMLGDINYRESFLEPYLRNELAHPVLSFAQLVSFTIFVPIVLMNLLIGLAVGDIAEVQKHASLKRIAMQVELHTSLEKKLPLWFLRKVDQKSTIVYPNKPRSGGMLFHIFCFLFCTGEIRQEIPNADKSLEMEILKQKYRLKDLTFLLEKQHELIKLIIQKMEIISETEDDDSHCSFQDRFKKEQMEQRNSRWNTVLRAVKAKTHHLEP

SEQ ID NO: 3 - protein, 5 aa, Homo sapiens
QRFSR

SEQ ID NO: 4 - protein, 5 aa, Rattus norvegicus
QHNPR

SEQ ID NO: 5 - protein, 5 aa, Unknown
  Description of Unknown SMR3 peptide
QRGPR